(19) United States
(12) Patent Application Publication
(10) Pub. No.: US 2023/0270956 A1
(43) Pub. Date: Aug. 31, 2023

(54) DOSE COUNTER ASSEMBLIES FOR MEDICAMENT DISPENSERS

(71) Applicant: LUPIN INC., Baltimore, MD (US)

(72) Inventors: Imran SHAIKH, Baltimore, MD (US); Mukul DALVI, Baltimore, MD (US); Xian Ming ZENG, Baltimore, MD (US); Cemeron ALBERG, Baltimore, MD (US); Tomas MATUSAITIS, Baltimore, MD (US)

(73) Assignee: LUPIN INC., Baltimore, MD (US)

(21) Appl. No.: 18/006,487

(22) PCT Filed: Jul. 21, 2021
(86) PCT No.: PCT/US2021/042625

§ 371 Date: Jan. 23, 2023

Related U.S. Application Data
(60) Provisional application No. 63/055,510, filed on Jul. 23, 2020.

(51) Int. Cl. A61M 15/00

(52) U.S. Cl. CPC A61M 15/0071; A61M 2202/064

(57) ABSTRACT

There is provided a dose counter assembly for use with a medicament dispenser, the dose counter assembly comprising: a) a units dose display means comprising a first count indicia and one or more driver teeth; b) a tens dose display means comprising a second count indicia; and c) a driver, wherein the first count indicia and second count indicia align at a common viewing area to collectively display a count sequence.

### FIELD

The present disclosure relates to dose counter assemblies for medicament dispensers and components for such dose counter assemblies.

### BACKGROUND

Medicaments are administered to a patient by inhalation using dry powder dispenser devices. Such devices are often referred to as dry powder inhalers (DPIs) and are used for the treatment and prophylaxis of respiratory diseases, including, but not limited to asthma, chronic obstructive pulmonary disease (COPD), bronchitis, lung infections, interstitial pulmonary fibrosis (IPF), pulmonary arterial hypertension (PAH), cystic fibrosis, Non-CF bronchiectasis, and lung transplant.

Generally described, dry powder inhalers carry medicament in reservoirs, capsules or blisters packages, and can otherwise carry medicament. The medicament carrier can include a blister strip containing a number of discrete doses of powdered medicament. Such devices can contain a mechanism accessing these doses, either piercing means or means to peel a lid sheet away from a base sheet. The powdered medicament can then be accessed and inhaled. Such a mechanism may also be used for dispensing medicament in tablets, capsules, pellets or pucks form wherein peeling away the lid sheet from the base sheet reveals a particular medicament form such as tablet for removal and subsequent consumption, particularly for oral administration.

One of the disadvantages arising from the use of such dry powder inhalers is that the user cannot readily determine the amount of medicament in the inhaler at any given time. In an extreme case this could mean that a user in need of a dose of medicament might find that the inhaler will not dispense a dose because its contents have already been exhausted. Therefore, it is useful for a user to know how many doses remain in the dispenser. To this end, a reliable dose counter device and methodology is desired, in order to register how many doses have been dispensed from the medicament dispenser and in order to inform a user how many more doses still remain to be dispensed.

Current regulatory guidance in some parts of the world recommends that a medicinal inhaler include a dose-counting device so that a patient may be aware when a device is nearing the end of its recommended number of actuations. It is desired that a dose counter be as nearly 100 percent reliable as possible and, in particular, that a device avoid undercounting. That is, the device should not deliver a dose without advancement of the counter, since this could lead a patient to believe there is more medication left in the device than actually remains.

U.S. Pat. No. 5,988,496 describes a dose counter comprising a first count wheel and second count wheel arranged to rotate about a common axis of rotation. The first count wheel includes a drive tongue that is movable between a rest position, in which it does not co-operate with the second count wheel and a drive position, in which it co-operates with the second count wheel to cause it to rotate about the common axis of rotation. The drive tongue is forced into position by action of a cam.

U.S. Pat. No. 8,113,199 discloses a dose counter for use with a medicament dispenser, said dose counter comprising a first count wheel arranged to rotate about a first axis of rotation, said first count wheel including count indicia and a set of primary drive teeth arranged annularly thereon for drivable rotation of the first count wheel about said first axis of rotation; a second count wheel arranged to rotate about the first axis of rotation, said second count wheel including count indicia and a set of secondary drive teeth arranged annularly thereon, and said second count wheel is arranged concentric to the first count wheel such that the respective count indicia are arranged concentrically; a kick wheel arranged to rotate about a second axis of rotation offset from the first axis of rotation, said kick wheel including a set of kick teeth arranged annularly thereon and in meshed relationship with the set of secondary drive teeth of the second count wheel such that rotary motion of the kick wheel results in rotary motion of the second count wheel; and, a drive wheel which is constantly in meshed relationship with the primary drive teeth of the first count wheel and which is adapted in use to couple with an operating mechanism of a medicament dispenser such that actuation of the operating mechanism will cause the drive wheel to rotate and to drive rotation of the first count wheel; wherein said first count wheel further includes a fixed index tooth arranged for intermittent meshing with the kick teeth of the kick wheel such that rotary motion of the kick wheel results from rotary motion of the first count wheel only when said intermittent meshing occurs; wherein the dose counter has a display region through which the count indicia of the first and second count wheels are rotatable; wherein the second count wheel comprises a shutter which is movable to a shuttering position in which it shutters the display region; wherein the dose counter is configured and arranged to display a count sequence in the display region with the count indicia and to cause the shutter to move to its shuttering position at the end of the count sequence; and wherein the dose counter is configured and arranged so that drive transmission from the first count wheel to the second count wheel through the kick wheel is disengaged when the shutter is in the shuttering position to keep the shutter in the shuttering position when the drive wheel drives further rotation of the first count wheel.

### SUMMARY

The inventors of the dose counter assemblies described herein have recognized a need in the art for a dose counter assembly, which is simple in operation, and provides accurate information of the dose to the user.

According to one aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver, wherein the first count indicia and second count indicia align at a common viewing area to collectively display a count sequence.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the units dose display means is arranged to rotate about a first axis of rotation and the tens dose display means is arranged to rotate about a second axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the units dose display means and the tens dose display means are non-coaxial.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means has a non-circular form.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means has a non-circular form; and wherein the units dose display means is arranged to rotate about a first axis of rotation and the tens dose display means is arranged to rotate about a second axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means has a polygonal form; and wherein the units dose display means is arranged to rotate about a first axis of rotation and the tens dose display means is arranged to rotate about a second axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia and a shutter; and c) a driver; wherein the tens dose display means has a polygonal form; and wherein the units dose display means is arranged to rotate about a first axis of rotation and the tens dose display means is arranged to rotate about a second axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia and a shutter; and c) a driver; wherein the tens dose display means has a polygonal form; wherein the tens dose display means is arranged non-concentric to the units dose display means.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia and a shutter; and c) a driver; wherein the tens dose display means has a pentagonal form; wherein the tens dose display means is arranged non-concentric to the units dose display means.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display wheel comprising a first count indicia and one or more driver teeth; b) a tens dose display wheel comprising a second count indicia; and c) a driver; wherein the units dose display wheel is arranged to rotate about a first axis of rotation, the tens dose display wheel is arranged to rotate about a second axis of rotation and the driver is arranged to rotate about a third axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display wheel comprising a first count indicia and one or more driver teeth; b) a tens dose display wheel comprising a second count indicia; and c) a driver; wherein the units dose display wheel and the tens dose display wheel are arranged to rotate about a common axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display wheel comprising a first count indicia and one or more driver teeth; b) a tens dose display wheel comprising a second count indicia and a shutter; and c) a driver; wherein the units dose display wheel and the tens dose display wheel are arranged to rotate about a common axis of rotation.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means is in the form of a slider.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means is arranged to move linearly.

Another aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver; wherein the tens dose display means is arranged to move linearly and tangent to the units dose display means.

According to one aspect of the disclosure, which can be combined with any other aspect, embodiment, or combination(s) of aspects and/or embodiments of the present disclosure, relates to a dose counter assembly for use with a medicament dispenser, the dose counter assembly comprising: a) a units dose display means comprising a first count indicia and one or more driver teeth; b) a tens dose display means comprising a second count indicia; and c) a driver, wherein the driver is engaged with the a units dose display means and wherein sufficient movement of the driver results in movement of the units dose display means, one or more driver teeth of the units dose display means being intermittently engaged, either directly or indirectly, with the tens dose display means such that movement of the tens dose display means results from movement of the units dose display means only when the intermittent engagement occurs.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### DETAILED DESCRIPTION

The present disclosure relates to a dose counter assembly for a medicament dispenser. Particularly, the present disclosure relates to a dose counter assembly for a medicament dispenser, such as a dry powder inhaler (DPI), and components of such a dose counter assembly.

In one of the embodiments, a dose counter assembly for use with a medicament dispenser, the dose counter assembly comprising:
a units dose display means comprising a first count indicia and one or more driver teeth;
a tens dose display means comprising a second count indicia; and
a driver,
wherein the first count indicia and second count indicia align at a common viewing area to collectively display a count sequence.

The dose counter assembly may include a units dose display means, a tens dose display means and a driver. The dose counter assembly may also optionally include a mount plate, a transfer wheel, a secondary driver, an idler gear, a shutter and a cover.

The units dose display means includes a first count indicia and displays the units place of the dose. The units dose display means can be of a circular shape or a non circular shape. In some embodiments, the units dose display means can take the form of a wheel. The units dose display wheel can include a set of teeth arranged annularly, preferably circumferentially thereon. The teeth can be therefore arranged in annular fashion at or about the circumference of the units dose display wheel. The units dose display wheel can further include one or more driver teeth (e.g., one, two, three, or four). In some embodiments, one or more driver teeth can be located on the circumference of the units dose display wheel. In some embodiments, one or more driver teeth can protrude outward from the circumference of the units dose display wheel. In some embodiments, the one or more driver teeth can be arranged for intermittent meshing with the teeth of the tens dose display means. The units dose display wheel may for example take the form of a disc or a ring. In some embodiments, the first count indicia are disposed on a disc that is affixed on the units wheel. The first count indicia may have a numbers such as ‘0’, ‘1’, ‘2’, ‘3’, ‘4’, ‘5’, ‘6’, ‘7’, ‘8’ and ‘9’. In some embodiments, the units dose display means can be driven by the driver. In some embodiments, the units dose display means can be constantly in meshed relationship with the driver.

In some embodiments, the units dose display means can be of polygonal shape which includes but not limited to triangular, square, rectangular, quadrilateral, pentagonal, hexagonal, heptagonal, octagonal, nonagonal, decagonal and the like.

In some embodiments, the units dose display means can be arranged to move rotationally. In some embodiments, the units dose display means can be arranged to rotate about a first axis of rotation.

The tens dose display means includes a second count indicia and displays the tens place of the dose. The tens dose display means can be of a circular shape or a non circular shape. In some embodiments, the tens dose display means can take the form of a wheel. The tens dose display wheel can include a set of teeth arranged annularly, preferably circumferentially thereon. The teeth can be therefore arranged in annular fashion at or about the circumference of the tens dose display wheel. The tens dose display wheel may for example, take the form of a disc or a ring. In some embodiments, the second count indicia are disposed on a disc that is affixed on the tens wheel. The second count indicia may have a numbers such as ‘0’, ‘1’, ‘2’, ‘3’, ‘4’, ‘5’, ‘6’, ‘7’, ‘8’ and ‘9’. In some embodiments, the tens dose display means can be driven by the units dose display means. The units dose display means can include one or more driver teeth arranged for intermittent meshing with the teeth of the tens dose display means. One or more driver teeth of the units dose display means may be brought into meshed relationship with the teeth of the tens dose display means on an intermittent basis. In some embodiments, the tens dose display means can be driven by the transfer wheel. The transfer wheel can include one or more driver teeth arranged for intermittent meshing with the teeth of the tens dose display means.

In some embodiments, the tens dose display means may be of polygonal shape which includes but not limited to triangular, square, rectangular, quadrilateral, pentagonal, hexagonal, heptagonal, octagonal, nonagonal, decagonal and the like.

In some embodiments, the tens dose display means can be arranged to move rotationally. In some embodiments, the tens dose display means can be arranged to rotate about a first axis of rotation. In some embodiments, the tens dose display means can be arranged to rotate about a second axis of rotation. In some embodiments, the tens dose display means is arranged non-concentric to the units dose display means. In some embodiments, the tens dose display means is arranged non-coaxial to the units dose display means.

In some embodiments, the units dose display means and the tens dose display means may be arranged to rotate about the same (for example common) axis of rotation. In some embodiments, the tens dose display means may be arranged co-axial to the units dose display means. In some embodiments, the tens dose display means may be arranged concentric to the units dose display means.

In some embodiments, the units dose display means may take the form of a ring and the tens dose display means (e.g. disc or ring shape) may be sized and shaped for receipt within the ring.

In some embodiments, the units dose display means and the tens dose display means may be arranged at the same level (for example, with same plane of rotation). In some embodiments, the units dose display means and the tens dose display means may be arranged at different levels (for example, with different planes of rotation).

In some of the embodiments, the units dose display means and the tens dose display means may be arranged to rotate in the same direction or in opposing directions (for example, one clockwise and one counter-clockwise).

In some embodiments, the tens dose display means can take the form of a slider. The tens dose display slider can include a set of teeth arranged linearly. The teeth can be therefore arranged in linear fashion at or about the underside surface of the tens dose display slider. The tens dose display means may for example take the form of a rectangle, an irregular rectangle or irregular quadrilateral or the like. In some embodiments, the second count indicia are disposed on the tens dose display slider in a linear fashion. In some embodiments, the tens dose display slider can be driven by the units dose display means. One or more driver teeth of the units dose display means may be brought into meshed relationship with the teeth of the tens dose display slider on an intermittent basis.

In some embodiments, the tens dose display means can be arranged to move linearly. The tens dose display means can be arranged to move linearly and tangent to the units dose display wheel. The tens dose display means can be arranged to move linearly over a rail of the mount plate.

The tens dose display means may optionally include a locking arm, a shutter, a post and a guide. In some embodiments, a shutter can be a portion which can be of any size, shape (e.g. square, rectangular, rectangular with rounded corners, quadrilateral and the like) and color (e.g. red, green, yellow and the like) which can extend along a portion of the tens dose display means. In some embodiments, the shutter can be a protrusion of tens dose display means. In some embodiments, the tens dose display means can include a colored portion and a protruding shutter. In some embodiments, the tens dose display means may have an end location where teeth are absent or removed. As a result of the absent teeth at the end location, the rotation of the units dose display means will not be transmitted to the tens dose display means. In other words, at the end location, the drive transmission from the units dose display means to the tens dose display means is disengaged. At the end location, the dose counter displays the shutter or reading ‘00’ in the common viewing area (viewing window) and remains at this position. The dispenser is now empty at the end of count sequence. The tens dose display means can include a post for driving the shutter. The shutter may be provided as a separate element of the dose counter or medicament dispenser or be formed as an integral part of the tens dose display means. In some embodiments, the shutter may be a separate component and may be driven by the post of the tens dose display means. At the end of count sequence, the shutter appears on the viewing window. The shutter may be locked in this position by a locking ridge of the cover. In some embodiments, the shutter overlies the first count indicia of the units dose display means.

In some embodiments, the dose counter assembly of the present disclosure can be arranged to count downwards (i.e. to count on from ‘n−1’ to ‘n’ on advancing), but that the counter assembly may be straightforwardly modified to count upwards (i.e. instead to count on from ‘n’ to ‘n+1’ on advancing).

In some embodiments, the first count indicia and the second count indicia may align at a common viewing area to collectively display a count sequence. The medicament dispenser housing may include a viewing window through which the count may be viewed.

The driver may rotate the units dose display means during each actuation of the medicament dispenser. The driver can include a set of teeth arranged annularly, preferably circumferentially thereon and one or more engaging slots. In some embodiments, the teeth of the driver can be in meshed relationship with the units dose display means teeth. In some embodiments, the driver and the units dose display means rotate in the same direction. In some embodiments, the driver can be arranged to rotate about the third axis of rotation. In some embodiments, the driver and the secondary driver may be arranged to rotate about the same (for example common) axis of rotation. In some embodiments, the driver can be adapted to couple with a dispensing mechanism of the medicament dispenser. In some embodiments, the driver can be driven by a component of the dispensing mechanism, for example, a first waste collector. Specific embodiments of the first waste collector and the dispensing mechanism that may be employed with the dose counter assembly of the present disclosure are described in a U.S. provisional patent application No. 62/994,307 by applicant that is incorporated herein by reference.

The mount plate can locate all components of the dose counter assembly. The mount plate can include an opening, a pawl and optionally a locking arm, optionally a rail, optionally one or more recesses and optionally one or more posts. The pawl may prevent the reverse movement of tens dose display means. The rail can be linear in shape. The mount plate can be of any shape including but not limited to square, rectangular, circular, polygonal and triangular and the like.

The dose counter assembly may further include a mechanism for locking the dose counter and/or the medicament dispenser. In some embodiments, the locking arm of the mount plate may come in contact with the locking key of tens dose display means at the end of the count sequence, which in turn lock the dose counter and/or medicament dispenser.

The transfer wheel may rotate the tens dose display means. The transfer wheel can include a set of teeth arranged annularly, preferably circumferentially thereon. The teeth can be therefore arranged in annular fashion at or about the circumference of the transfer wheel. The transfer wheel may further include one or more driver teeth (e.g., one, two, three, or four). In some embodiments, one or more driver teeth can be located on the circumference of the transfer wheel. In some embodiments, the one or more driver teeth of the transfer can be arranged for intermittent meshing with the teeth of the tens dose display means. The transfer wheel may for example, take the form of a disc or a ring. In some embodiments, the transfer wheel can be driven by the units dose display means. In some embodiments, the transfer wheel can be driven by a secondary driver.

In some embodiments, the transfer wheel can be arranged to move rotationally. In some embodiments, the transfer wheel can be arranged to rotate about a third axis of rotation. In some embodiments, the transfer wheel can be located eccentric to the units dose display means and the tens dose display means.

The dose counter assembly may count the number of doses left to be taken or the number of doses taken. The dose counter assembly may be mechanical (e.g., non-electronic) or non-mechanical (e.g., electronic, digital) in form.

The dose counter assembly can be used with a medicament dispenser of any suitable type, including, but not limited, to a dry powder inhaler (DPI), a pressurized metered-dose inhaler (pMDI), a mist inhaler and a nebulizer. Specific embodiments of the medicament dispensers that may be employed with the dose counter assembly of the present disclosure are described in a U.S. provisional patent application No. 62/994,307 by applicant that is incorporated herein by reference.

The medicament dispenser can include a housing for locating the dose counter assembly and other components of the dose dispensing mechanism. The housing can include a base cover and a top cover for enclosing the dose counter assembly, one or more medicament carriers, an inner housing assembly, a base plate, and components of the dispensing mechanism. The housing can also include a viewing window through which the count may be viewed. In one aspect, a shutter is provided to close off the viewing window at a predetermined point, particularly at the ‘ end of life’ of the medicament dispenser, which typically corresponds to the point at which all doses in the normal delivery cycle have been provided. In aspects, the shutter may be provided as a separate element of the dose counter assembly or medicament dispenser or be formed as an integral part of the tens dose display means, as described hereinbefore.

The dose counter assembly described herein can be used with a medicament dispenser for dispensing medicament from at least one single elongate form medicament carrier, such an elongate form medicament carrier commonly existing in the art as blister pack packaging or strip packaging (but which could incorporate any multiple dose medicament carrier wherein the carrier houses multiple, distinctly separate medicament doses in a form suited for being housed within the dispenser), said dispenser comprising: a) a dispensing mechanism comprising: i) a driver, ii) a carrier engagement, iii) an advancer, iv) a flow director, v) a first waste collector for collecting a first waste stream, vi) a second waste collector for collecting a second waste stream; b) a medicament access body; and c) a cover for the medicament access body.

The dispenser can receive a single or plural (e.g., two, three, or four) medicament carriers carrying multiple distinct medicament doses. The medicament carrier can be an elongated sheet comprising a base sheet, such as a base sheet comprising distinct dose storage areas, e.g., blisters, and a lid sheet providing a sealed means by which the individual doses are held within the dose storage areas (e.g., blisters) of the base sheet, as in the medicament carrier can be a blister strip medicament carrier. Access to the medicament(s) comprised within the pockets of the blister strip is by any suitable access means including piercing, tearing or peeling apart the relevant blister pockets. According to certain aspects, wherein a medicament carrier or medicament carriers within the dispenser is or are blister strips, the first waste stream can be the base sheet of the blister strip, and a second waste stream can be the lid sheet of the blister strip.

The medicament dispenser has a dispending mechanism for dispensing the distinct medicament doses carried by the medicament carrier for administration for inhalation by the patient. The dispensing mechanism can include a driver, a carrier engagement, an advancer, a first waste collector for collecting a first waste stream, a second waste collector for collecting a second waste stream, and a flow director.

A driver can be provided for driving the actuation of the dispensing mechanism. According to certain embodiments, the driver can actuate the dispensing mechanism in cooperation with movement of the cover of the medicament access body, the driver being movably engaged with the medicament access body cover in such a way that movement of the medicament access cover can result in movement of the driver. In turn, the driver can be movably engaged with the advancer.

A carrier engagement can be provided for advancing the distinct medicament dose portions of the medicament carrier. An advancer can be provided for rotating the carrier engagement. The registration of a count by the dose counter assembly can be responsive to advancement by the carrier engagement of a distinct medicament dose from the medicament carrier.

The first waste collector can be provided for collecting the first waste stream, e.g., the base sheet of a used blister strip medicament carrier. Further, the first waste collector is capable of advancing the driver of the dose counter assembly. In further aspects, the first waster collector can include one or more pins into which the engaging slots of the driver can be placed. In some embodiments, the first waster collector advances the driver of the dose counter assembly. The registration of a count by the dose counter assembly can be responsive to advancement by the first waste collector.

The second waste collector can be provided for collecting the second waste stream, e.g., the lid sheet of a used blister strip medicament carrier, can aid in peeling apart the base sheet and lid sheet of each such medicament carrier to open or otherwise access the blister pocket to make the dose stored therein available for administration. Further, the second waste collector is capable of providing tension to and maintaining tension on the waste stream being collected, e.g., the lid sheet.

The flow director can be provided for guiding airflow towards one or more available medicament doses, e.g., medicament doses within opened blister pocket(s) of a blister strip medicament carrier, for releasing the powder contained therein; and subsequently guiding the liberated powder to a medicament access body, e.g., mouthpiece, for inhalation by a patient.

The medicament dispenser comprises a dose counter assembly for counting each time a distinct medicament dose of the medicament carrier is advanced by said carrier engagement. Suitably, advancing by means of the carrier engagement results either directly or indirectly in drivable rotation of the units dose display means of the dose counter assembly herein.

The medicament dispenser may further provided with means to manipulate, and in particular magnify, an analogue count indicium. The means may in one embodiment, comprise a magnifying window. In another embodiment, the means comprises a prismatic viewer capable of acting on an indicium and causing it to be displayed in manipulated form at a desired viewing position.

The components of the dose counter assembly and medicament dispenser can be made from the material of constructions, including, but not limited to, acrylonitrile butadiene styrene (ABS), polycarbonate/acrylonitrile butadiene styrene terpolymer blend (PC/ABS), Polyoxymethylene (POM), nylon and silicone rubber.

Whilst the dose counter assembles herein have been mainly illustrated for use with a medicament dispenser in which an elongate form blister strip is advanced to enable release of medicament from the individual blisters thereof the dose counter is also suitable for use with other types of medicament dispenser. Thus, the dose counter assembly is also suitable for use with metered dose inhaler (MDI) type devices in which, generally actuation is responsive to an actuating movement (e.g. push down the MDI canister) relative to its housing; reservoir dry powder inhalers (RDPI) and reservoir liquid spray inhalers (RLSI) in which, generally metering is responsive to an metering movement (e.g. bring metering cavity into communication with the bulk reservoir) relative to the bulk reservoir; and other types of multi dose dry powder inhalers (MDPI) in which, generally dose advancement to a delivery position is responsive to a dose advancement movement (e.g. advancing a blister pack to move the next blistered dose to the delivery position) relative to a housing. Suitable medicament dispensers herein are for the dispensing of medicament, particularly for the treatment and prophylaxis of respiratory diseases, including, but not limited to asthma, chronic obstructive pulmonary disease (COPD), bronchitis lung infections, interstitial pulmonary fibrosis (IPF), pulmonary arterial hypertension (PAH), cystic fibrosis, Non-CF bronchiectasis or lung transplant

The medicament dispenser is designed to dispense one or more medicaments contained therein. Such a medicament or number of medicaments can be any one or more medicament(s) housed within one or more medicament carrier(s) capable of being received by and housed within the medicament dispenser.

According to certain aspects, which can be combined with any other aspect, embodiment, or combination(s) of aspects and/or embodiments, the medicament dispenser is designed to receive an elongate form medicament carrier carrying multiple distinct medicament dose portions. In one aspect, which can be combined with any other aspect, embodiment, or combination(s) of aspects and/or embodiments, the medicament dispenser can receive a single elongate form medicament carrier. In another aspect, the dispenser can receive plural elongate form medicament carriers. In yet another aspect, the dispenser can receive one to four elongate form medicament carriers, such as one, two, three, or four elongate form medicament carriers. In one aspect, which can be combined with any other aspect, embodiment, or combination(s) of aspects and/or embodiments, the dispenser can receive two elongate form medicament carriers. In certain embodiments, which can be combined with any other aspect, embodiment, or combination(s) of aspects and/or embodiments, the medicament dispenser can receive a single elongate form medicament carrier which houses one, two, three, four, or more different medicaments, each housed in distinct dose housing or dose storage areas (e.g., blister pockets), one or more such pockets being positioned such that they are accessible when the elongate medicament carrier is appropriately positioned within the dispenser. According to alternative aspects, which can be combined with any other aspect, embodiment, or combination(s) of aspects and/or embodiments, the medicament dispenser can receive a plurality (e.g., two, three, or four) elongate form medicament carriers which can optionally house one, two, three, four, or more different medicaments, each medicament dose being housed in distinct dose housing or dose storage areas (e.g., blister pockets) however one or more being positioned such that they are accessible substantially simultaneously, that is at about the same time, along with one or more dose storage areas, of one or more other medicament carriers held within the dispenser. In certain aspects, the medicament dispenser can advance a plurality of medicament carriers substantially simultaneously. In certain other aspects, the medicament dispenser can advance a plurality of medicament carriers sequentially, e.g., such that there is no substantial overlap or no detectable overlap in movement of two or more medicament carrier engagement mechanisms in operation.

According to certain embodiments, which can be combined with any other aspect, embodiment, or combination(s) of aspects and/or embodiments, the elongate form medicament carrier can be any elongate form medicament carrier that houses a plurality of discrete doses of medicament in a sequence, such that when positioned in an appropriate position for access by the medicament dispenser a single dose of medicament within the carrier is available for administration and additional discrete doses of medicament are subsequently made available for administration upon further actuation and positioning. Such successive availability of different medicament dosages provides the dispenser with the ability to provide successive dosings of one or more medicaments, such as multiple dose-per-day administration of one or more medicaments, or sequential daily administration of one or more medicaments, and the like.

In some embodiments, medicament housed within one or more medicament carrier comprises one or more active pharmaceutical ingredients (APIs) and optionally, one or more pharmaceutically acceptable carriers. The active pharmaceutical ingredients and pharmaceutically acceptable carriers may be present in micronized form, non-micronized form, or mixtures thereof.

According to some embodiments, the medicament within each of the medicament storage areas of any single medicament carrier is the same. In alternative embodiments, the medicament within at least one of the medicament storage areas is different from the medicament within any at least one other medicament storage area within the same carrier. In certain aspects, the medicament within at least one of the medicament storage areas of a first carrier varies from the medicament held within at least one of the medicament storage areas of a second carrier.

According to certain aspects of the present invention, the dose portions of medicament within any single medicament carrier are all the same. In alternative aspects, at least one of the individual doses of medicament within any single medicament carrier is different from at least one other separated individual dose of medicament within the same single medicament carrier. According to yet further aspects, at least one of the individual doses of medicament within any one medicament carrier is different from at least one of the individual doses of medicament within any second or more medicament carriers.

According to one aspect of the present invention, one or more active pharmaceutical ingredients (APIs) that can be used in the inventions selected from, analgesics, e.g., codeine, dihydromorphine, ergotamine, fentanyl or morphine; anginal preparations, e.g., diltiazem; antiallergics, e.g., cromoglycate (e.g., as the sodium salt), ketotifen or nedocromil (e.g., as the sodium salt); anti-infectives, e.g., cephalosporins, penicillins, streptomycin, sulphonamides, tetracyclines and pentamidine, particularly tobramycin, aztreonam, colistin, ceftazidime, gentamycin, levofloxacin, ciprofloxacin, amikacin, vancomycin, rifampin and isoniazid; mucolytics e.g., N-acetylcysteine, carbocysteine, erdosteine, and dornase alfa.

According to embodiments, the medicaments used in the device can include agents for treating idiopathic pulmonary fibrosis e.g., nintedanib and pirfenidone; antihistamines, e.g., methapyrilene; anti-fungals, e.g., Pulmazole and voriconazole; anti-inflammatories, e.g., beclomethasone (e.g., as the dipropionate ester), fluticasone (e.g., as the propionate or furoate ester), flunisolide, budesonide, rofleponide, mometasone e.g., as the furoate ester), ciclesonide, triamcinolone (e.g., as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g., as free base or sulphate), levoalbuterol, procaterol, salmeterol (e.g., as xinafoate), ephedrine, adrenaline, fenoterol (e.g., as hydrobromide), formoterol (e.g., as fumarate), arformoterol, clenbuterol, olodaterol, indacaterol, vilanterol, isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g., 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g., as maleate); α4 integrin inhibitors, e.g., (2S)-3-[4-({[4-(aminocarbonyl)-1-pipe ridinyl]carbonyl}oxy)phenyl]-2-[((2S-)-4-methyl-2-{[2-(2-methylphenoxy)acetyl]amino}pentanoyl)amino]propanoic acid (e.g., as free acid or potassium salt), diuretics, e.g., amiloride; anticholinergics, e.g., ipratropium (e.g., as bromide), tiotropium, umeclidinium, aclidinium, glycopyrronium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament.

In some embodiments, the medicament can be a mono-therapy (for example, single active medicament containing) product, or it may be a combination therapy (for example, plural active medicaments containing) product. In certain aspects, the medicament carrier can contain mono-active medicament dose portions or multi-active medicament dose portions (i.e., two or more than two APIs). Suitable medicaments or medicament components of a combination therapy product are typically selected from the group consisting of anti-inflammatory agents (for example a corticosteroid particularly inhaled corticosteroid (ICS) or an NSAID), anticholinergic agents (for example, an M₁, M₂, M₁/M₂ or M₃ receptor antagonist particularly long-acting muscarinic antagonist (LAMA), other β₂-adrenoreceptor agonists particularly long-acting β₂-agonist (LABA), anti-infective agents (e.g., an antibiotic or an antiviral), and antihistamines. All suitable combinations are envisaged. The present invention, in some embodiments, also provides combination therapy products, such as double or triple LAMA, LABA, and/or ICS combinations.

According to certain aspects, suitable anti-inflammatory agents include corticosteroids and NSAIDs. Suitable corticosteroids are those oral and inhaled corticosteroids and their pro-drugs which have anti-inflammatory activity. Examples of such agents include but are not limited to include methyl prednisolone, prednisolone, dexamethasone, fluticasone propionate, fluticasone furoate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g., the 17-propionate ester or the 17,21-dipropionate ester), budesonide, flunisolide, mometasone esters (e.g., the furoate ester), triamcinolone acetonide, rofleponide, ciclesonide, butixocort propionate, RPR-106541, and ST-126. In some aspects, preferred corticosteroids include fluticasone propionate, fluticasone furoate 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

According to some embodiments, suitable NSAIDs include cromolyns (e.g., sodium cromoglycate, nedocromil sodium), phosphodiesterase (PDE) inhibitors (e.g., theophylline, PDE4 inhibitors or mixed PDE3/PDE4 inhibitors), leukotriene antagonists, inhibitors of leukotriene synthesis, iNOS inhibitors, tryptase and elastase inhibitors, β₂integrin antagonists and adenosine receptor agonists or antagonists (e.g., adenosine 2a agonists), cytokine antagonists (e.g., chemokine antagonists) or inhibitors of cytokine synthesis. Suitable other β₂-adrenoreceptor agonists include salmeterol (e.g., as the xinafoate), salbutamol (e.g., as the sulphate or the free base), formoterol (e.g., as the fumarate), vilanterol (e.g., as the trifenatate), indacaterol (e.g., as the maleate), fenoterol or terbutaline and salts thereof.

In some aspects, suitable phosphodiesterase 4 (PDE4) inhibitors include compounds that are known to inhibit the PDE4 enzyme or which are discovered to act as a PDE4 inhibitor, and which are only PDE4 inhibitors, not compounds which inhibit other members of the PDE family as well as PDE4. According to certain aspects, it may be preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4), and the other form of this catalytic site which binds rolipram with a high affinity is denominated the “high affinity” binding site (HPDE 4). This term, “HPDE4,” should not be confused with the term “hPDE4,” which is used to denote human PDE4.

A method for determining IC₅₀s ratios is set out in U.S. Pat. No. 5,998,428, which is incorporated herein in full by reference as though set out herein. See also PCT application WO 00/51599 for another description of the said assay.

In some embodiments, suitable PDE4 inhibitors include those compounds which have a salutary therapeutic ratio, for example, compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that in some aspects, the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC50 ratio of about 0.1 or greater; said ratio is the ratio of the IC₅₀ value for competing with the binding of 1 nM of [³H]R-rolipram to a form of PDE4 which binds rolipram with a high affinity over the IC₅₀ value for inhibiting the PDE4 catalytic activity of a form which binds rolipram with a low affinity using 1 muM[³M]-cAMP as the substrate.

In some embodiments, suitable agents are those PDE4 inhibitors that have an IC₅₀ ratio of greater than 0.5, and particularly those compounds having a ratio of greater than 1.0. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low-affinity binding site and which have an IC₅₀ ratio of 0.1 or greater.

Other suitable medicament compounds include, according to certain embodiments: cis-4-cyano-4-[3-(cyclopentyloxy)-4-methoxyphenyl]cyclohexane-1-carboxylic acid (also known as cilomalast) disclosed in U.S. Pat. No. 5,552,438 and its salts, esters, pro-drugs or physical forms; AWD-12-281 from elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a, 10b-hexahydro-methoxy-8-methoxy-2-methylbenzo[c] [1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

According to certain embodiments, suitable anticholinergic agents are those compounds that act as antagonists at the muscarinic receptor, in particular, those compounds, which are antagonists of the M1 and M2 receptors. Exemplary compounds include but may not be limited to the alkaloids of the belladonna plants as illustrated by the likes of atropine, scopolamine, homatropine, hyoscyamine; these compounds are normally administered as a salt, being tertiary amines.

In some aspects, particularly suitable anticholinergics can include ipratropium (e.g., as the bromide), sold under the name Atrovent, umeclidinium (e.g., as the bromide), aclidinium (e.g., as the bromide), glycopyrronium (e.g., as the bromide), oxitropium (e.g., as the bromide) and tiotropium (e.g., as the bromide) (CAS-139404-48-1). Also of interest in some embodiments are methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118.

According to some aspects, suitable antihistamines (also referred to as H₁-receptor antagonists) include any one or more of the numerous antagonists known which inhibit H₁-receptors and are safe for human use. All such agents can be reversible, competitive inhibitors of the interaction of histamine with H1-receptors. Examples of such agents include ethanolamines, ethylenediamines, and alkylamines. In addition, other first-generation antihistamines include those which can be characterized as based on piperizine and phenothiazines Second generation antagonists, which are non-sedating, have a similar structure-activity relationship in that they retain the core ethylene group (the alkylamines) or mimic the tertiary amine group with piperizine or piperidine. Exemplary antagonists are as follows but may not be limited to: Ethanolamines: carbinoxamine maleate, clemastine fumarate, diphenylhydramine hydrochloride, and dimenhydrinate. Ethylenediamines: pyrilamine amleate, tripelennamine HCl, and tripelennamine citrate. Alkylamines: chlropheniramine and its salts such as the maleate salt, and acrivastine. Piperazines: hydroxyzine HCl, hydroxyzine pamoate, cyclizine HCl, cyclizine lactate, meclizine HCl, and cetirizine HCl. Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁ receptor antagonist that may be used in combination with a PDE4 inhibitor. Particularly suitable anti-histamines in some aspects include methapyrilene and loratadine. In respect of combination products, co-formulation compatibility is generally determined on an experimental basis by known methods and may depend on the chosen type of medicament dispenser action.

According to certain embodiments, the medicament components of a combination product administered by a dispenser described herein are suitably selected from the group consisting of anti-inflammatory agents (for example a corticosteroid or an NSAID), anticholinergic agents (for example, an M₁, M₂, M₁/M₂ or M₃ receptor antagonist), other β₂-adrenoreceptor agonists, anti-infective agents (e.g., an antibiotic or an antiviral), and antihistamines. All suitable combinations are envisaged.

In certain aspects, the co-formulation compatible components comprise a β₂-adrenoreceptor agonist and a corticosteroid; and the co-formulation incompatible component comprises a PDE-4 inhibitor, an anti-cholinergic or a mixture thereof. The β₂-adrenoreceptor agonists may, for example, be salbutamol (e.g., as the free base or the sulphate salt) or salmeterol (e.g., as the xinafoate salt) or formoterol (e.g., as the fumarate salt). The corticosteroid may for example, be a beclomethasone ester (e.g., the dipropionate) or a fluticasone ester (e.g., the propionate) or budesonide.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic (e.g., ipratropium bromide or tiotropium bromide) or a mixture thereof.

In another example, the co-formulation compatible components comprise budesonide and formoterol (e.g., as the fumarate salt), and the co-formulation incompatible component comprises a PDE-4 inhibitor, an anti-cholinergic (e.g., ipratropium bromide or tiotropium bromide) or a mixture thereof.

Medicament dispensers, as disclosed herein, have a variety of structural configurations and can be used for dispensing powders, tablets, capsules, pellets or pucks, or mixtures thereof for nasal, pulmonary or oral administration. In particular, the medicament dispensers can be used to dispense a powder comprising one or more active pharmaceutical ingredient(s)(API) and optionally, one or more micronized or non-micronized pharmaceutically acceptable carrier(s) and/or excipient(s), wherein the dispensing mechanism comprises of three steps to operate the medicament dispenser: open, dispense and close.

Generally, powdered medicament particles suitable for delivery to the bronchial or alveolar region of the lung have an aerodynamic diameter of less than about 10 micrometers, preferably less than about 6 micrometers (e.g., about 0.5-10 micrometers, about 0.5-6 micrometers, about 0.5-5 micrometers, about 0.5-3 micrometers, about 1-10 micrometers, about 1-6 micrometers, or about 1-5 micrometers). Other sized particles may be used if delivery to other portions of the respiratory tract is desired, such as the nasal cavity, mouth, or throat. In some aspects, the medicament administered via a dispenser of the present invention can be delivered as a pure drug, but in some aspects, it can be more appropriate or preferable that medicaments are delivered together with one or more excipients or carriers which are suitable for inhalation. Suitable excipients or carriers include organic excipients such as polysaccharides (for example starch, cellulose and the like), lactose, trehalose, leucine, trileucine, pentaleucine and other polyleucines, glucose, mannitol, amino acids, magnesium stearate and maltodextrins, surfactants such as 1,2-Distearoyl-sn-glycero-3-phosphocholine (DSPC), 1,2-Distearoyl-sn-glycero-3-phosphoethanolamine (DSPE), perfluorocarbon such as perflubron (perfluorooctyl bromide) and inorganic excipients such as calcium carbonate, calcium chloride, magnesium chloride or sodium chloride. In a particular embodiment, carrier or excipient is lactose.

Particles of powdered medicament and/or excipient may be produced by conventional techniques, for example by micronization, milling or sieving. Additionally, medicament and/or excipient powders may be engineered with particular densities, size ranges, or characteristics. Particles may comprise active agents, surfactants, wall forming materials, or other components considered desirable by those of ordinary skill.

### BRIEF DESCRIPTION OF THE DRAWINGS

The following figures and related description thereof are incorporated to explain better and exemplify the invention described herein, without limiting its scope.

Some elements of the embodiments of the dispenser or its components are exemplified in a particular form and, accordingly, are sometimes described in such exemplary embodiments with a descriptive name in connection with the description of the embodiments shown in the figures that follow below. To aid the reader in understanding the various exemplary embodiments of the invention described in connection with figures, the following description of several such terms is provided here:

The use of the term “meshed” is commonly used within the description of figures to describe a movable engagement between two components, e.g., which is present when two gear-like mechanisms interface with each other.

The driver is exemplified as a “driver gear”.

The units dose display means is exemplified as a “units dose display wheel” or “units wheel”

The tens dose display means is exemplified as a “tens dose display wheel” or “tens wheel” or “tens dose display slider” or “tens slider”.

As noted above, this description of terms used in connection with the embodiments described below in connection with the figures is intended to aid the reader in understanding such exemplary aspects of the invention and should not be interpreted as limiting the scope of the invention in any way. Additional aspects of the invention described in connection with the figures will also be discussed below, but generally using terminology already introduced in this disclosure, such as the first waste capture component.

FIG. 1 shows a diagrammatic representation of a dose counter assembly constituting a first embodiment of the disclosure.

FIG. 2 shows an exploded view of the dose counter assembly with its components according to the first embodiment of the present disclosure along with the axis of rotation of the components.

FIGS. 3 to 5 show a working mechanism of the dose counter assembly according to the first embodiment of the present disclosure.

FIG. 6 shows a diagrammatic representation of a units dose display wheel according to the first embodiment of the present disclosure.

FIG. 7 shows a diagrammatic representation of a driver gear according to the first embodiment of the present disclosure.

FIG. 8 shows a diagrammatic representation of a mount plate according to the first embodiment of the present disclosure.

FIG. 9 shows a diagrammatic representation of a tens dose display wheel according to the first embodiment of the present disclosure.

FIG. 10 shows a locking mechanism of the dose counter assembly according to the first embodiment of the present disclosure.

FIG. 11 shows a diagrammatic representation of a dose counter assembly constituting a second embodiment of the disclosure.

FIG. 12 shows an exploded view of the dose counter assembly with its components according to the second embodiment of the present disclosure along with the axis of rotation of the components.

FIGS. 13 to 15 show a working mechanism of the dose counter assembly according to the second embodiment of the present disclosure.

FIG. 16 shows a diagrammatic representation of a units dose display means according to the second embodiment of the present disclosure.

FIG. 17 shows diagrammatic representation of a topside view of a tens dose display means according to the second embodiment of the present disclosure.

FIG. 18 shows a diagrammatic representation of an underside view of the tens dose display means according to the second embodiment of the present disclosure.

FIG. 19 shows a locking mechanism of the dose counter assembly according to the second embodiment of the present disclosure.

FIG. 20 shows a diagrammatic representation of a dose counter assembly constituting a third embodiment of the disclosure.

FIG. 21 shows an exploded view of the dose counter assembly with its components according to the third embodiment of the present disclosure along with the axis of rotation of the components.

FIGS. 22 to 24 show a working mechanism of the dose counter assembly according to the third embodiment of the present disclosure.

FIG. 25 shows a diagrammatic representation of a topside view of a tens dose display means according to the third embodiment of the present disclosure.

FIG. 26 shows a diagrammatic representation of an underside view of the tens dose display means according to the third embodiment of the present disclosure.

FIG. 27 shows a diagrammatic representation an end location of the dose counter assembly according to the third embodiment of the present disclosure.

FIG. 28 shows a diagrammatic representation of a dose counter assembly constituting a fourth embodiment of the disclosure.

FIG. 29 shows an exploded view of the dose counter assembly with its components according to the fourth embodiment of the present disclosure along with the axis of rotation of the components.

FIGS. 30 to 32 show a working mechanism of the dose counter assembly according to the third embodiment of the present disclosure.

FIG. 33 shows a diagrammatic representation of a topside view of a units dose display wheel according to the fourth embodiment of the present disclosure.

FIG. 34 shows a diagrammatic representation of an underside view of the units dose display wheel according to the fourth embodiment of the present disclosure.

FIG. 35 shows a diagrammatic representation of a driver gear according to the fourth embodiment of the present invention.

FIG. 36 shows diagrammatic representation of the mount plate according to the fourth embodiment of the present disclosure.

FIG. 37 shows a diagrammatic representation of a topside view of the tens dose display wheel according to the fourth embodiment of the present disclosure.

FIG. 38 shows a diagrammatic representation of an underside view of the tens dose display wheel according to fourth embodiment of the present disclosure.

FIG. 39 shows a locking mechanism according to the fourth embodiment of the present disclosure.

FIG. 40 shows a diagrammatic representation of a dose counter assembly constituting a fifth embodiment of the disclosure.

FIG. 41 shows an exploded view of the dose counter assembly with its components according to the fifth embodiment of the present disclosure along with the axis of rotation of the components.

FIGS. 42 to 44 show a working mechanism of the dose counter assembly according to the fifth embodiment of the present disclosure.

FIG. 45 shows a diagrammatic representation of the mount plate according to the fifth embodiment of the present disclosure.

FIG. 46 shows a diagrammatic representation of a secondary driver according to the fifth embodiment of the present disclosure.

FIG. 47 shows a diagrammatic representation of a topside view of a driver gear and its components according to the fifth embodiment of the present disclosure.

FIG. 48 shows a diagrammatic representation of an underside view of the driver gear according to the fifth embodiment of the present disclosure.

FIG. 49 shows a diagrammatic representation of a transfer wheel according to the fifth embodiment of the present disclosure.

FIG. 50 shows a diagrammatic representation of a topside view of the units dose display wheel according to the fifth embodiment of the present disclosure.

FIG. 51 shows a diagrammatic representation of an underside view of the units dose display wheel according to the fifth embodiment of the present disclosure.

FIG. 52 shows a diagrammatic representation of a topside view of the tens dose display wheel according to fifth embodiment of the present disclosure.

FIG. 53 shows a diagrammatic representation of an underside view of the tens dose display wheel in according to fifth embodiment of the present disclosure.

FIGS. 54 to 55 show a locking mechanism of the dose counter assembly according to the fifth embodiment of the present disclosure.

FIG. 56 shows a diagrammatic representation of a dose counter assembly constituting a sixth embodiment of the disclosure.

FIG. 57 shows an exploded view of the dose counter assembly with its components according to the sixth embodiment of the present disclosure along with the axis of rotation of the components.

FIGS. 58 to 60 show a working mechanism of the dose counter assembly according to the sixth embodiment of the present disclosure.

FIG. 61 shows a diagrammatic representation of the mount plate according to the sixth embodiment of the present disclosure.

FIG. 62 shows a diagrammatic representation of a driver gear according to the sixth embodiment of the present disclosure.

FIG. 63 shows a diagrammatic representation of a topside view of a units dose display means according to the sixth embodiment of the present disclosure.

FIG. 64 shows a diagrammatic representation of an underside view of the units dose display wheel according to the sixth embodiment of the present disclosure.

FIG. 65 shows a diagrammatic representation of a tens dose display means according to the sixth embodiment of the present disclosure.

FIG. 66 shows a locking mechanism of the dose counter assembly according to the sixth embodiment of the present disclosure.

FIG. 67 shows a diagrammatic representation of a dose counter assembly constituting a seventh embodiment of the disclosure.

FIG. 68 shows an exploded view of the dose counter assembly with its components according to the seventh embodiment of the present disclosure along with the axis of rotation of the components.

FIGS. 69 to 71 show a working mechanism of the dose counter assembly according to the seventh embodiment of the present disclosure.

FIG. 72 shows a diagrammatic representation of the mount plate according to the seventh embodiment of the present disclosure.

FIG. 73 shows a diagrammatic representation of a topside view of the ten dose display means according to the seventh embodiment of the present disclosure.

FIG. 74 shows a diagrammatic representation of an underside view of the ten dose display means according to the seventh embodiment of the present disclosure.

FIG. 75 shows a diagrammatic representation of a topside view of the units dose display means according to the seventh embodiment of the present disclosure.

FIG. 76 shows a diagrammatic representation of an underside view of the units dose display means according to the seventh embodiment of the present disclosure.

FIG. 77 shows a locking mechanism of the dose counter assembly according to the seventh embodiment of the present disclosure.

FIG. 78 shows a partially exploded view of the medicament dispenser with the dose counter assembly of the present disclosure.

FIG. 79 shows a diagrammatic representation of a topside view of an embodiment of the dispensing mechanism of the medicament dispenser.

FIG. 80 shows a diagrammatic representation of an embodiment of the first waste collector that is compatible with the dose counter assembly and the medicament dispenser described herein.

FIGS. 81A to 81C show a demonstration of the advancement of the dose count in the viewing window of the medicament dispenser.

### DETAILED DESCRIPTION OF EXEMPLARY EMBODIMENTS

To better illustrate aspects of the invention, particular embodiments and their operation will be described in this section, often with reference to the devices and components shown in the Figures. It is intended that the scope of the present invention herein disclosed should not be limited by any particular embodiment described herein. While various embodiments of the present invention have been described above, it should be noted that they have been presented by way of example only, and not limitation. Numerous changes to the disclosed embodiments can be made in accordance with the disclosure herein without departing from the spirit or scope of the invention. Further, any single embodiment or aspect described within this disclosure can be combined with any one or more other herein described aspects or embodiments, the description of such combinations considered to be incorporated herein as if such a combination had been described in detail.

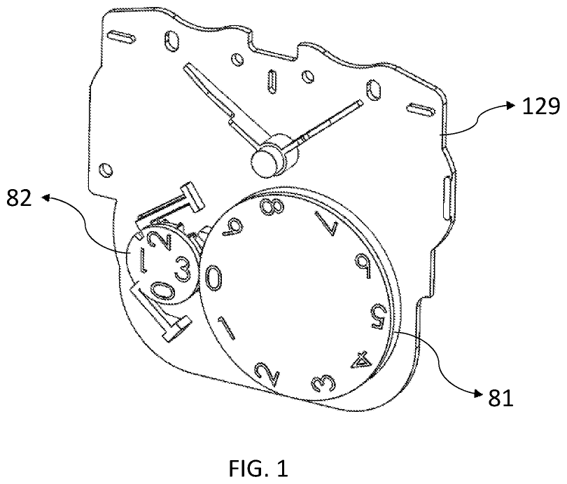

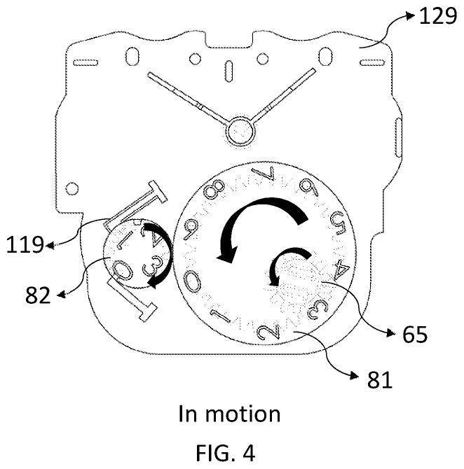

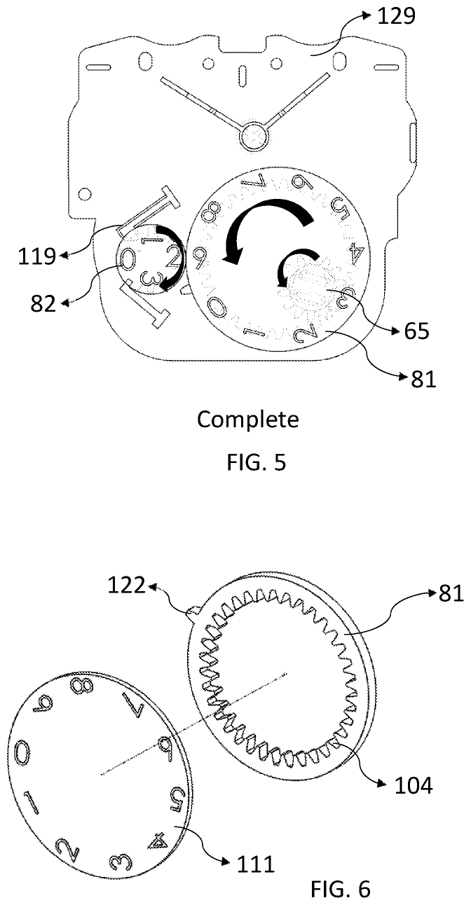

A FIRST embodiment of the dose counter assembly is shown in FIGS. 1 to 10 of the accompanying drawings.

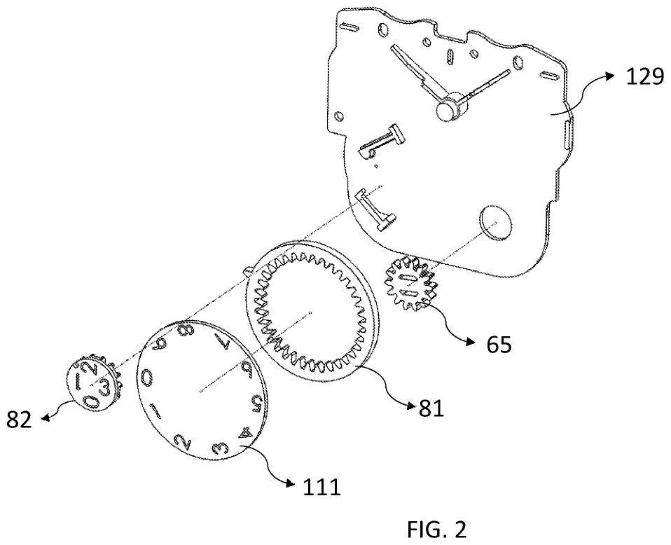

FIG. 1 shows a perspective view of the dose counter assembly. The units wheel (81) and the tens wheel (82) are located on the mount plate (129). FIG. 2 shows an exploded view of the dose counter assembly with its components along with the axis of rotation. The components include the units wheel (81), the tens wheel (82) and the driver gear (65). The units dose indicia are disposed on a disc (111) that can be affixed on the units wheel (81). The units wheel (81) can rotate at the first axis of rotation, the tens wheel (82) can rotate at the second axis of rotation and the driver gear (65) can rotate at the third axis of rotation. The driver gear (65) rotates and increments the units wheel (81) during each actuation of the dispenser. Both the driver gear (65) and the units wheel (81) may rotate in same direction. The driver tooth (122) of the units wheel (see FIG. 6) drives the tens wheel (82) on every ten actuations of the dispenser. The units wheel (81) and tens wheel (82) may rotate in the opposite direction. The units wheel (81) and tens wheel (82) collectively display the dose count on the viewing window (48) of the dispenser. The pawl (119) prevents the reverse rotation of the tens wheel (82).

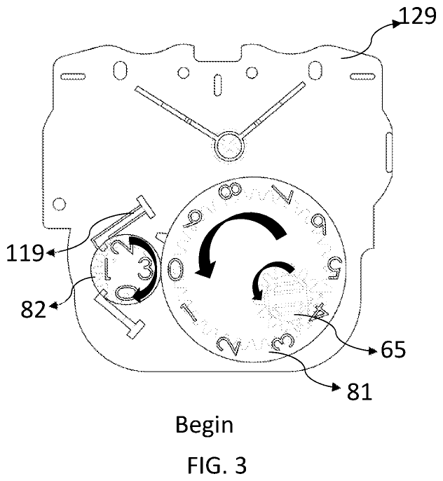

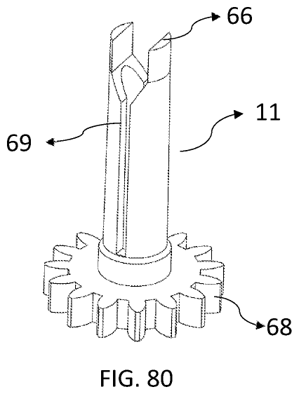

FIGS. 3 to 5 show working of the dose counter assembly and the movement of the dose indicia according to the first embodiment of the present disclosure in sequence of begin, in motion and complete. The dose counter assembly can be driven by the first waste collector (11) (see FIG. 80). The medicament dispenser cover (3) is opened (rotated) in response to the user action causing the other components of the dispensing mechanism, the first waste collector (11) and the driver gear (65) to rotate. The first waste collector (11) rotates in the counter clockwise direction resulting in counter clockwise motion of the driver gear (65) and the units wheel (81). The driver gear (65) and the units wheel (81) are arranged such that when the user opens (rotates) the cover (3), the units wheel (81) rotates and a single indicium thereon is advanced (for example the ‘units’ count moves on one unit).

Where the previous visible count was x0 (e.g. 30 or 20 or 10), the counting action resulting from the use operation is subtly different. Once again, the cover (3) is rotated in response to user action causing the driver gear (65) to rotate and in turn rotation of the units wheel (81) and the ‘unit’ indicium moves on from ‘0’ to ‘9’. This rotation of the units wheel (81) however, also brings the one or more driver teeth (122) into meshed relationship with the teeth (105) of the tens wheel (82) such that the units wheel (81) rotates in the counter clockwise direction and in turn, the tens wheel (81) rotates in the clockwise direction. As shown in FIGS. 3 to 5, the gearing of the relevant wheels 65, 81 and 82 is arranged such that the tens wheel (82) rotates and a single indicium thereon is advanced from ‘3’ to ‘2’ (for example the ‘tens’ count moves down exactly one unit).

When the user closes (rotates) the cover (3), the ratcheting mechanism of the dispensing mechanism and/or pawl (119) prevents the rotation of the of the dose counter assembly in the opposite direction.

It will be appreciated that the above usage of the counter has been described in terms of a dose counter assembly arranged to count downwards, but that the counter assembly may be straightforwardly modified to count upwards.

FIG. 6 shows an embodiment of the units wheel (81). The units wheel (81) may include teeth (104) on its inner circumference and a driver tooth (122). The units wheel driver tooth (122) engages with the tens wheel teeth (105) after every ten actuations of the dispenser. The units dose indicia are disposed on a disc (111) that can be affixed on the units wheel (81). The units wheel (81) rotates every discrete angle during each actuation of the dispenser to display the units integer of the dose in the viewing window (48).

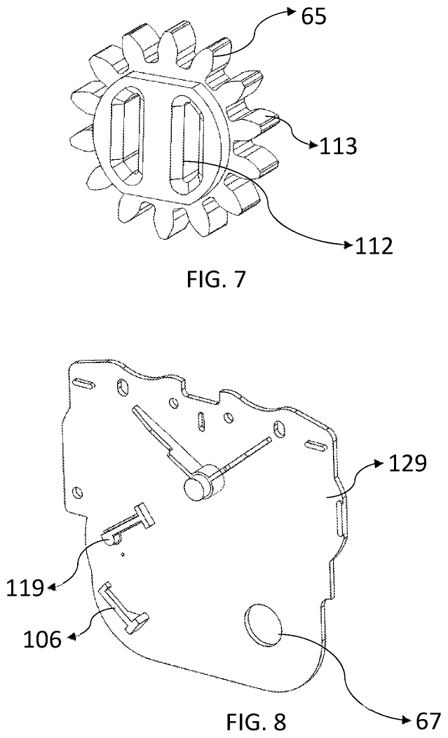

FIG. 7 illustrates the driver gear (65) which may include teeth (113) and engaging slots (112). The driver gear (65) can be driven by the first waste collector (11) (see FIG. 80) via the driver gear engaging slots (112). The driver gear (65) rotates in one direction during each complete actuation of the dispenser and does not rotate in the opposite direction when the dispenser is being closed after use. The unit wheel teeth (104) can always be in a meshed relationship with the driver gear teeth (113). The units wheel (81) can be driven by the driver gear via teeth (113).

FIG. 8 illustrates an aspect of the mount plate (129). The components of the dose counter assembly can be located on the mount plate (129). The pawl (119) and the locking arm (106) can be integral to the mount plate (129). The opening (67) allow the first waste collector pins (60) to pass through the mount plate (129) and rotate the driver gear (65) via the engaging slots (112).

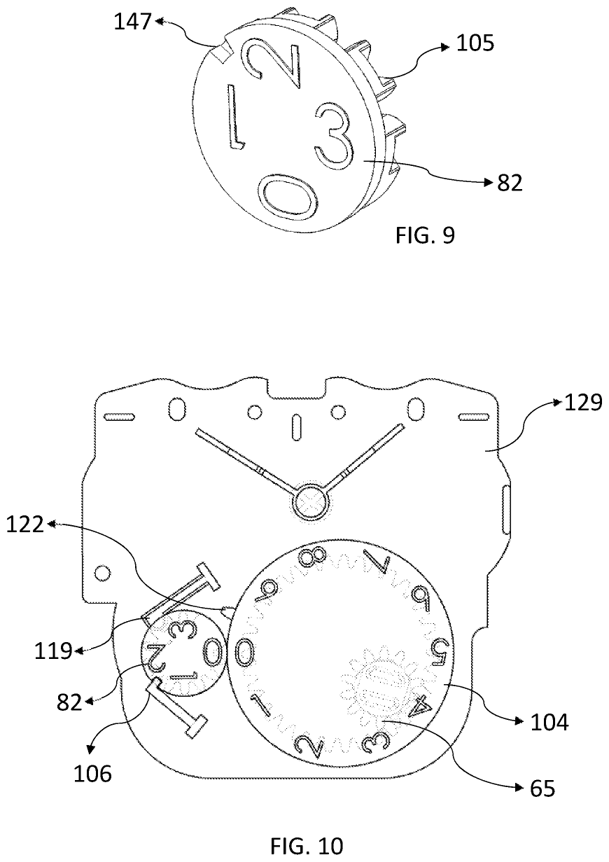

FIG. 9 illustrates an aspect of the tens wheel (82). The tens wheel (82) may include teeth (105) on its underside and a locking key (147) on the topside. The tens dose indicia are disposed on the tens wheel (81). The tens wheel (82) can be driven by the units wheel driver tooth (122) after every ten actuations of the dispenser. The locking key (147) comes in contact with the locking arm (106) of the mount plate at the end of count sequence and the dose count displays ‘00’ in the viewing window (48).

FIG. 10 shows an aspect of the locking mechanism of the dose counter and the dispenser. After the end of dispenser life when all doses are exhausted, the locking key (147) of the tens wheel comes in contact with the mount plate the locking arm (129) and locks the dose counter and the dispensing mechanism of the dispenser. At this position, the dose counter displays ‘00’ in the viewing window (48).

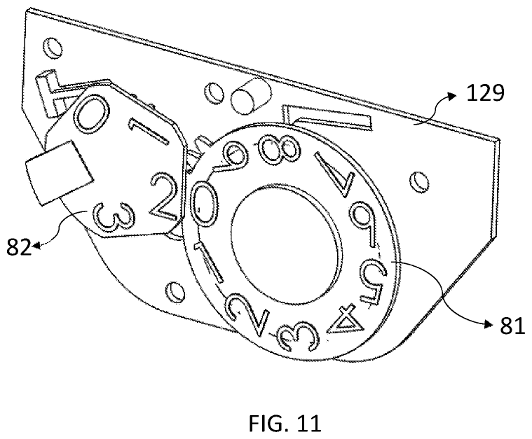

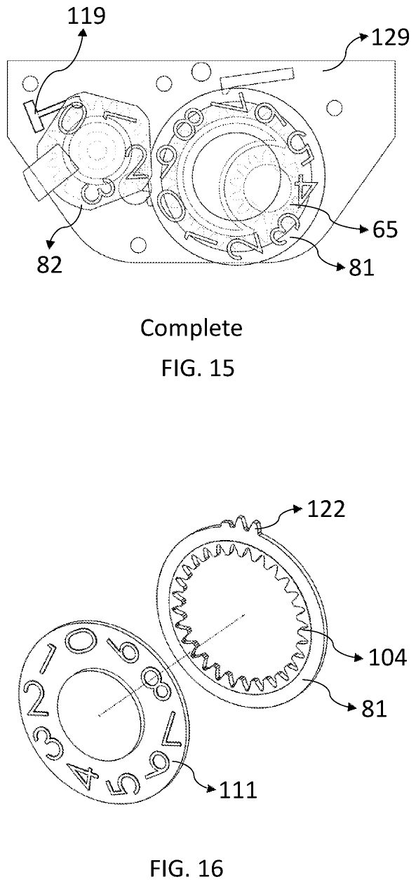

A SECOND embodiment of the dose counter assembly is shown in FIGS. 11 to 19 of the accompanying drawings.

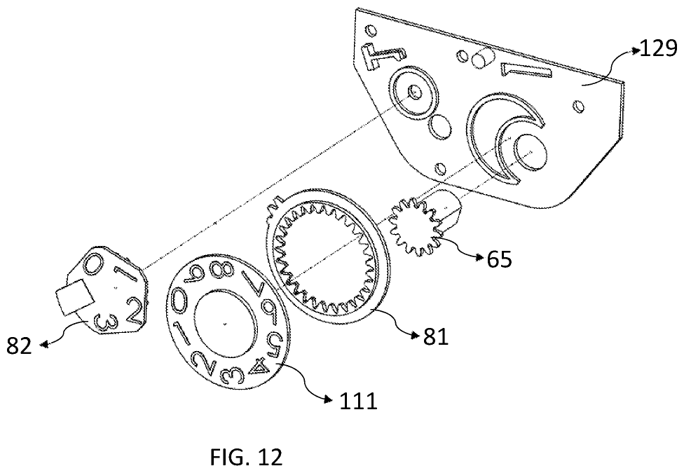

FIG. 11 shows a perspective view of the dose counter assembly. The units wheel (81) and a polygonal tens wheel (82) are located on the mount plate (129). FIG. 12 shows an exploded view of the dose counter assembly with its components along with the axis of rotation. The components include the units wheel (81), the tens wheel (82) and the driver gear (65). The units dose indicia are disposed on a disc (111) that can be affixed on the units wheel (81). The units wheel (81) can rotate at the first axis of rotation, the tens wheel (82) can rotate at the second axis of rotation and the driver gear (65) can rotate at the third axis of rotation.

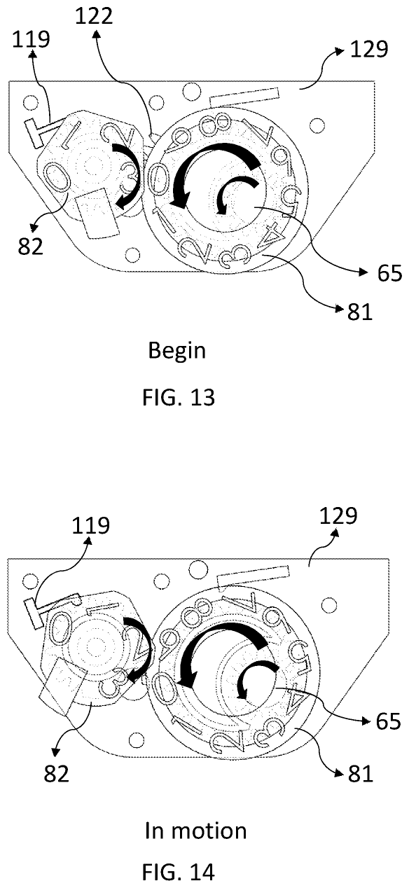

FIGS. 13 to 15 show working of the dose counter assembly and the movement of the dose indicia according to second embodiment of the present disclosure in sequence of begin, in-motion and complete. The driver gear (65) rotates and increments the units wheel (81) during each actuation of the dispenser. Both the driver gear (65) and the units wheel (81) may rotate in same direction. The driver teeth (122) of the units wheel (see FIG. 13) drives the tens wheel (82) on every ten actuations of the dispenser. The units wheel (81) and tens wheel (82) may rotate in the opposite direction. The units wheel (81) and tens wheel (82) collectively display the dose count in the viewing window (48) of the dispenser. The pawl (119) prevents the reverse rotation of the tens wheel (82). The movements of the units and tens indicia are same as described for the first embodiment of the present disclosure.

FIG. 16 shows another embodiment of the units wheel (81). The units wheel (81) may include teeth (104) on its inner circumference and driver teeth (122). The units wheel driver teeth (122) engage with the tens wheel teeth (105) after every ten actuations of the dispenser. The units dose indicia are disposed on a disc (111) that can be affixed on the units wheel (81). The units wheel (81) rotates every discrete angle during each actuation of the dispenser to display the units integer of the dose in the viewing window.

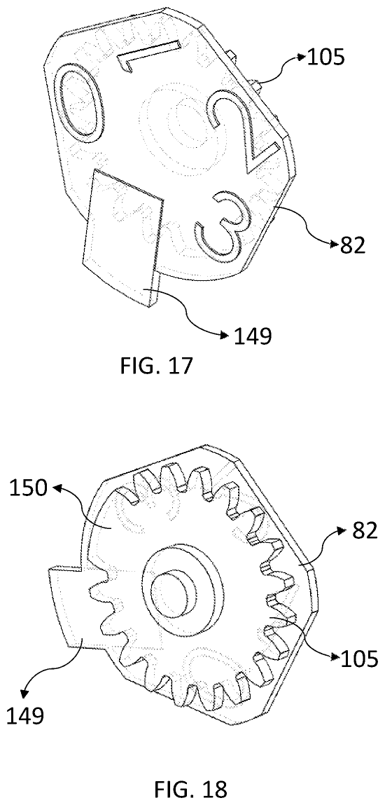

FIGS. 17 and 18 illustrate topside and underside of the tens wheel (82) according to another aspect of the disclosure. The tens dose indicia are disposed on the topside of the tens wheel (82). The tens wheel (82) may also include the shutter (149) on its topside and teeth (105) on the underside. The tens wheel (82) may also include an end location wherein the teeth (105) are removed or absent. The tens wheel (82) can be driven by the units wheel driver teeth (122) after every ten actuations of the dispenser. At the end of count sequence, the shutter (149) appears in the viewing window (48). Once the shutter (149) appears in the viewing window, the units wheel gear teeth (122) abuts with the end location (150) which in turn blocks the rotation of units wheel. In other words, the units wheel gear teeth (122) cannot rotate the tens wheel (82) due to absence of teeth at the tens wheel the end location (150).

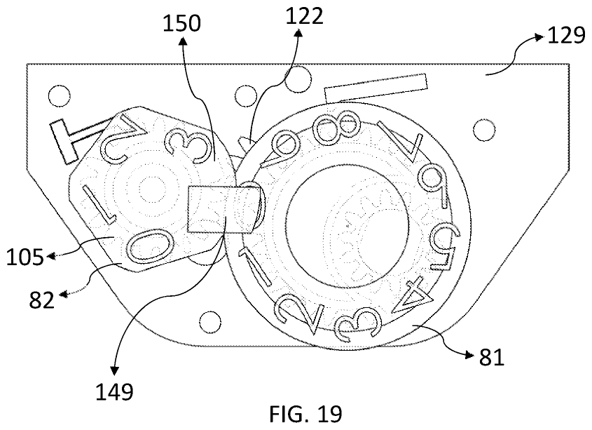

FIG. 19 shows an aspect of the locking mechanism of the dose counter and the dispenser. After the end of dispenser life when all doses are exhausted, the shutter (149) appears in the viewing window (48). At this position, the units wheel gear teeth (122) cannot rotate the tens wheel (82) due to absence of teeth at the tens wheel the end location (150) and locks the dose counter and dispensing mechanism of the dispenser.

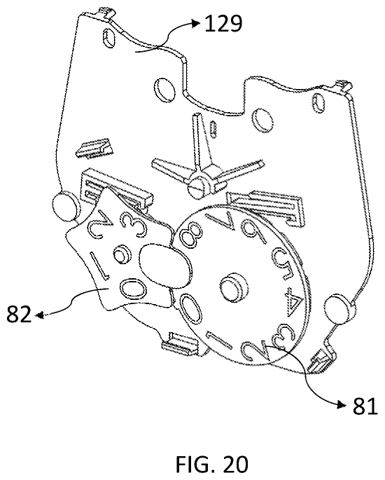

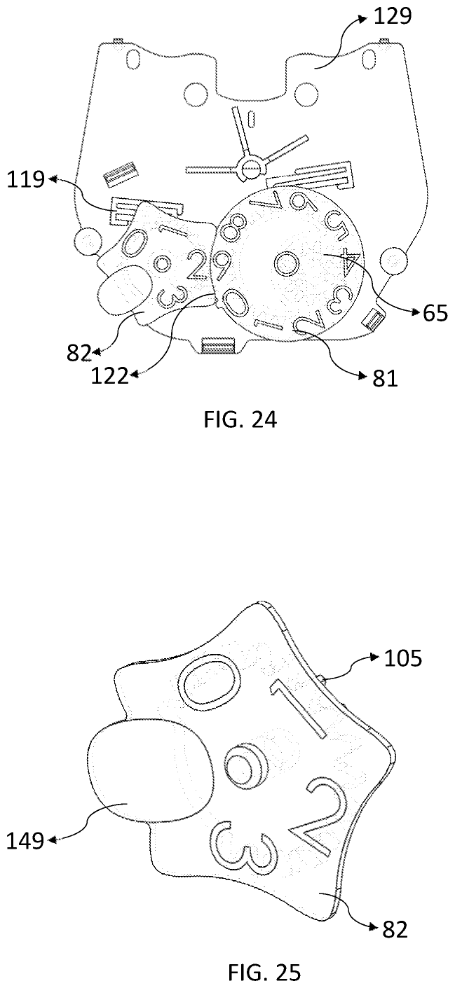

A THIRD embodiment of the dose counter assembly is shown in FIGS. 20 to 27 of the accompanying drawings.

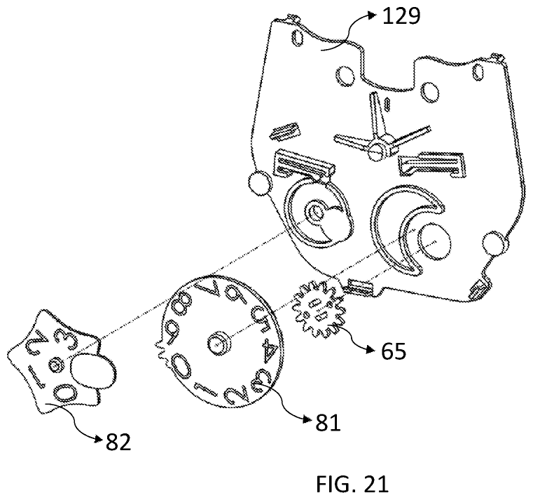

FIG. 20 shows a perspective view of the dose counter assembly. The units wheel (81) and a pentagonal tens wheel (82) are located on the mount plate (129). FIG. 21 shows an exploded view of the dose counter assembly with its components along with the axis of rotation. The components include the units wheel (81), the tens wheel (82) and the driver gear (65). The units dose indicia are disposed on the units wheel (81). The units wheel (81) can rotate at the first axis of rotation, the tens wheel (82) can rotate at the second axis of rotation and the driver gear (65) can rotate at the third axis of rotation. The units wheel (81) may include teeth (104) on its inner circumference and driver teeth (122). The units wheel (81) rotates every discrete angle during each actuation of the dispenser to display the units integer of the dose in the viewing window (48).

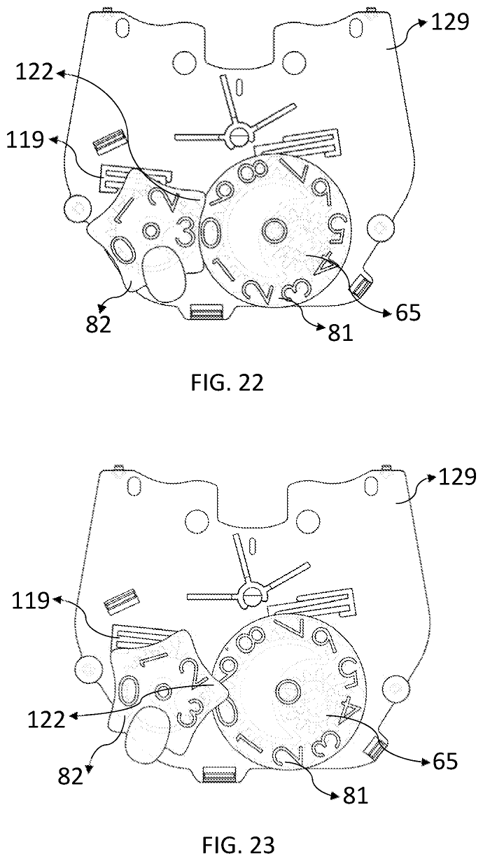

FIGS. 22 to 24 show working of the dose counter assembly and the movement of the dose indicia according to the third embodiment of the present disclosure in sequence of begin, in-motion and complete. The driver gear (65) rotates and increments the units wheel (81) during each actuation of the dispenser. Both the driver gear (65) and the units wheel (81) may rotate in same direction. The driver teeth (122) of the units wheel (see FIG. 21) engage with the tens wheel teeth (105) after every ten actuations of the dispenser. The units wheel (81) and tens wheel (82) may rotate in the opposite direction. The units wheel (81) and tens wheel (82) collectively display the dose count on the viewing window (48) of the dispenser. The pawl (119) prevents the reverse rotation of the tens wheel (82). The movements of the units and tens indicia are same as described for the first embodiment of the present disclosure.

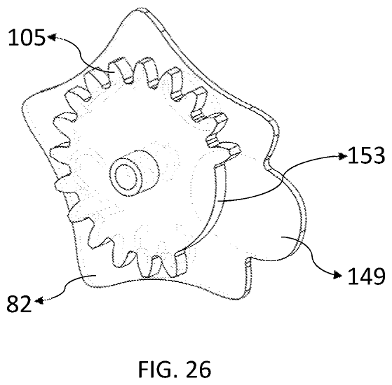

FIGS. 25 and 26 illustrate the topside and underside of the tens wheel (82) according to another aspect of the disclosure. The tens dose indicia are disposed on the topside of the tens wheel (81). The tens wheel (82) may also include the shutter (149) on its topside and teeth (105) on the underside. The tens wheel (82) may also include an end location wherein the teeth (105) are removed or absent. The tens wheel (82) can be driven by the units wheel driver teeth (122) after every ten actuations of the dispenser. At the end of count sequence, the shutter (149) appears in the viewing window (48). Once the shutter (149) appears in the viewing window, the units wheel driver teeth (122) and the tens wheel teeth (105) no longer mesh. The units wheel driver teeth (122) cannot rotate the tens wheel (82) due to absence of the teeth the end location (153) of the tens wheel. In other words, the drive transmission from the units dose display wheel to the tens dose display wheel is disengaged.

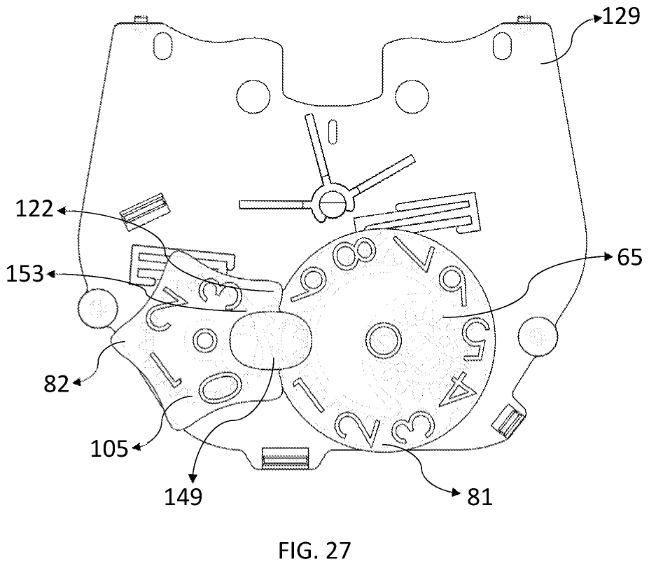

FIG. 27 shows an aspect of the end location of the dose counter. After the end of dispenser life when all doses are exhausted, the shutter (149) appears in the viewing window (48). At this position, the units wheel driver teeth (122) cannot rotate the tens wheel (82) due to absence of teeth at the tens wheel end location (153). When user actuates the dispenser, the units wheel (81) continues to rotate without rotating the tens wheel (82) and the tens wheel shutter (149) remains at this position in the viewing window (48).

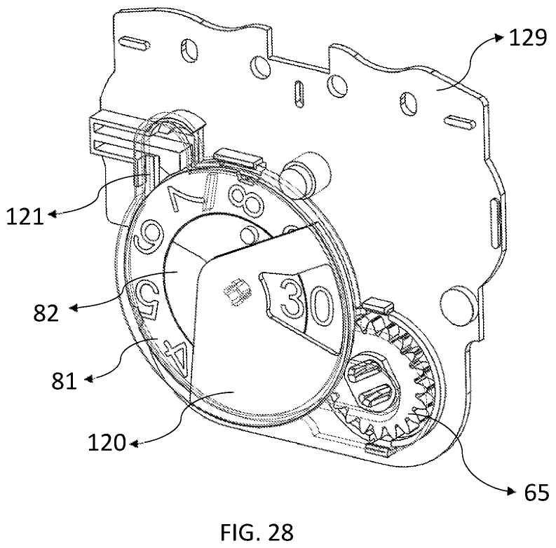

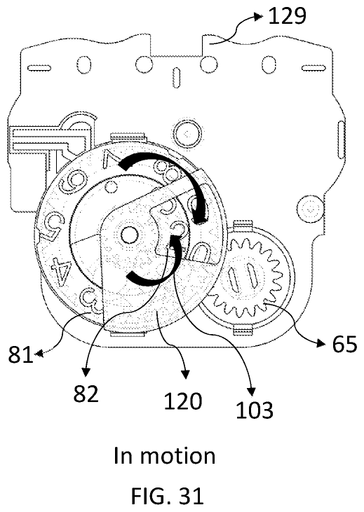

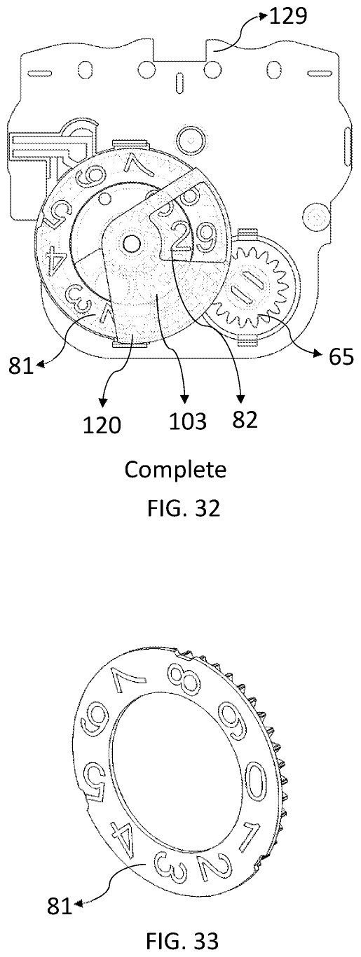

A FOURTH embodiment of the dose counter assembly is shown in FIGS. 28 to 39 of the accompanying drawings.

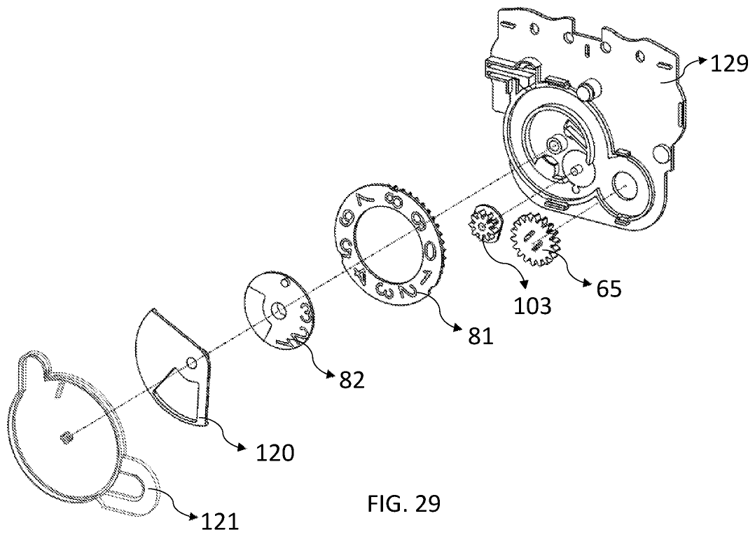

FIG. 28 shows a perspective view of the dose counter assembly. The units wheel (81), the tens wheel (82) and the driver gear (65) are located on the mount plate (129). FIG. 29 shows an exploded view of the dose counter assembly with its components along with the axis of rotation. The components include the units wheel (81), the tens wheel (82), the driver gear (65), the transfer wheel (103), the shutter (120) the cover (121). The units dose indicia are disposed on the units wheel (81). The units wheel (81) and the tens wheel (82) can rotate at the first axis of rotation, the transfer wheel (103) can rotate at the second axis of rotation and the driver gear (65) can rotate at the third axis of rotation.

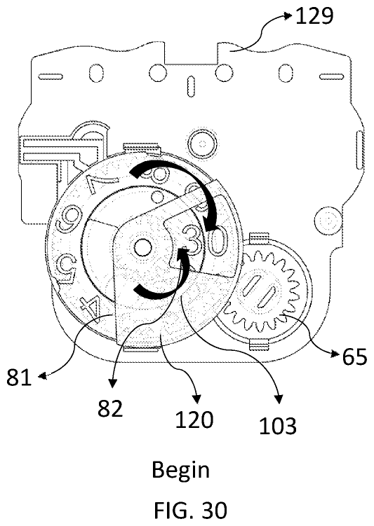

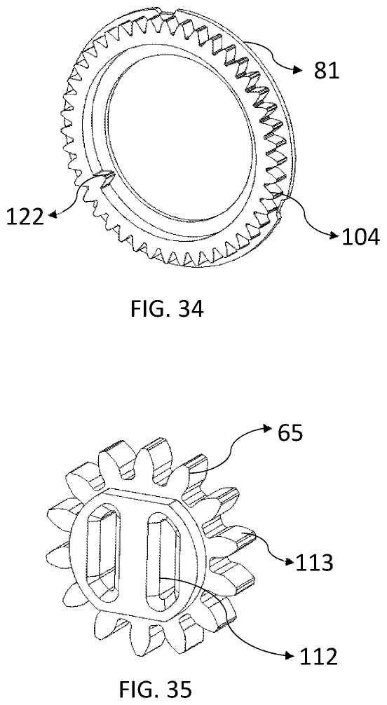

FIGS. 30 to 32 show working of the dose counter assembly and the movement of the dose indicia according to fourth embodiment of the present disclosure in sequence of begin, in-motion and complete. The units wheel (81) and the tens wheel (82) are concentric and rotate about the post (134). The driver gear (65) rotates and increments the units wheel (81) during each actuation of the dispenser. The tens wheel (82) can be driven by the transfer wheel (103). The driver tooth (122) of the units wheel (see FIG. 34) drives the transfer wheel (103) on every ten actuations of the dispenser. The units wheel (81) and tens wheel (82) may rotate in the opposite direction. The units wheel (81) and tens wheel (82) collectively display the dose count on the viewing window (48) of the dispenser. The pawl (119) prevents the reverse rotation of the tens wheel (82). The shutter (120) can be driven by the post (135) located on the tens wheel (82). The shutter (120) remains in the viewing window (48) when the dose count reaches ‘00’.

FIGS. 33 and 34 illustrate the topside and underside of the units wheel (81) according to another aspect of the disclosure. The units dose indicia are disposed on the topside of the units wheel (81). The units wheel (81) may include teeth (104) circumferentially and driver tooth (122) on its underside surface. The teeth (104) and the driver tooth (122) may be extended in the opposite direction. The units wheel teeth (104) are constantly in meshed relationship with the driver gear teeth (113). The units wheel (81) can be driven by the driver gear via gear teeth (113). The units wheel (81) rotates every discrete angle during each actuation of the dispenser to display the units integer of the dose in the viewing window. The units wheel driver tooth (122) comes in contact with the transfer wheel (103) after every ten actuations of the dispenser and thereby rotates the transfer wheel (103).

FIG. 35 illustrates the driver gear (65) which may include teeth (113) and engaging slots (112). The driver gear (65) can be driven by the first waster collector (11) (see FIG. 80) via the driver gear engaging slots (112). The driver gear (65) rotates in one direction during each complete actuation of the dispenser and does not rotate in the opposite direction when the dispenser is being closed after use. The driver gear (65) rotates during each actuation of the dispenser and thereby rotates the units wheel (81).

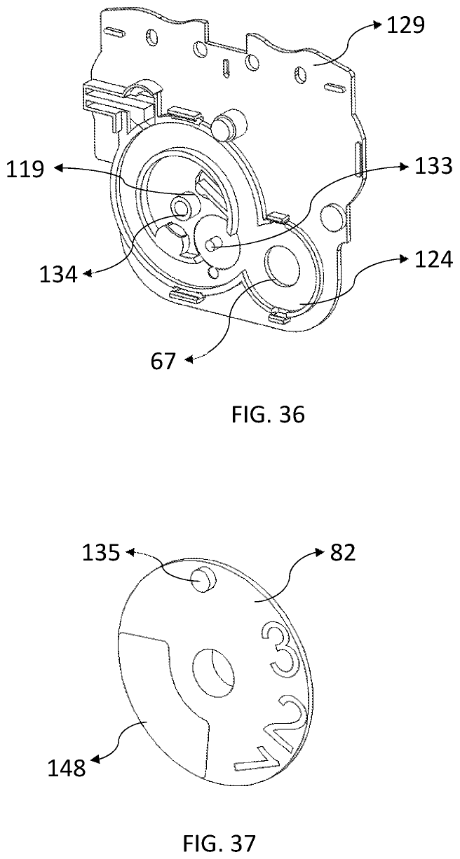

FIG. 36 illustrates an aspect of the mount plate (129). The components of the dose counter assembly can be located on the mount plate (129). The pawl (119) can be integral to the mount plate (129) and prevents the reverse rotation of the tens wheel (82). The opening (67) allow the first waste collector pins (60) to pass through the mount plate (129) and rotate the driver gear (65) via the engaging slots (112). The recess (124) can locate the driver gear (65). The transfer wheel (103) can rotate about the stud (133). The units wheel (81) and the tens wheel (82) can be concentric and rotate about the post (134).

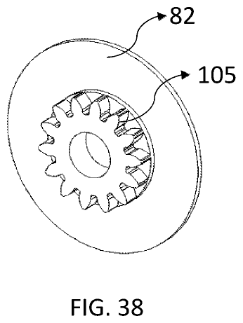

FIGS. 37 and 38 illustrate the topside and underside of the tens wheel (82) according to another aspect of the disclosure. The tens dose indicia are disposed on the topside of the tens wheel (81). The tens wheel (82) may also include the colored portion (148) and the post (135) on its topside and teeth (105) on the underside. The tens wheel (82) can be constantly in meshed relationship with transfer wheel (103) and thereby driven by the transfer wheel (103) after every ten actuations. The shutter (120) can be driven by the post (135) located on the tens wheel (82).

When the user actuates the dispenser, the driver gear (65) drives and increments the units wheel (81). After every ten actuations of the dispenser, the units wheel driver tooth (122) meshes and rotates the transfer wheel (103). The transfer wheel (103) which is constantly in meshed relationship with the tens wheel (82) rotates the tens wheel (82).

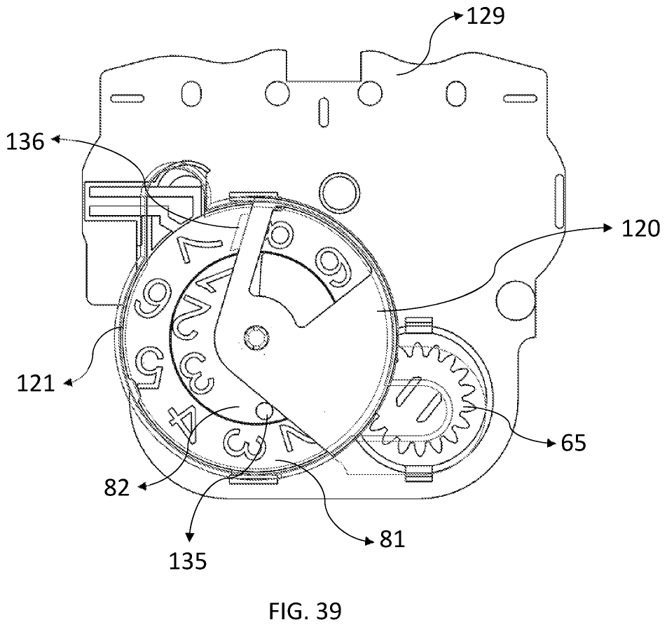

FIG. 39 shows an aspect of the locking mechanism of the dose counter and the dispenser. After the end of dispenser life when all doses are exhausted, the shutter (120) appears in the viewing window (48). The shutter (120) is locked at this position by the locking ridge (136) located on the dose counter cover (121) and thereby locks the entire dose counter mechanism along with the dispensing mechanism of the dispenser.

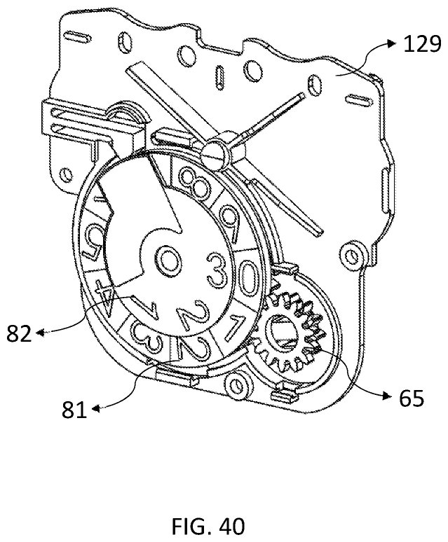

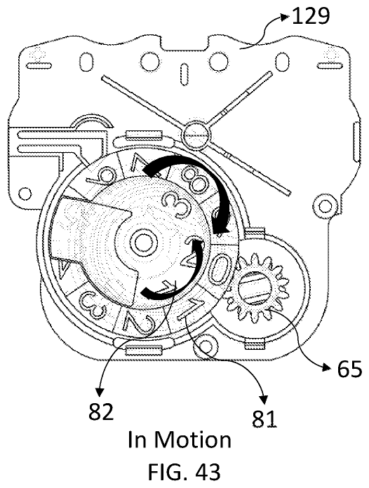

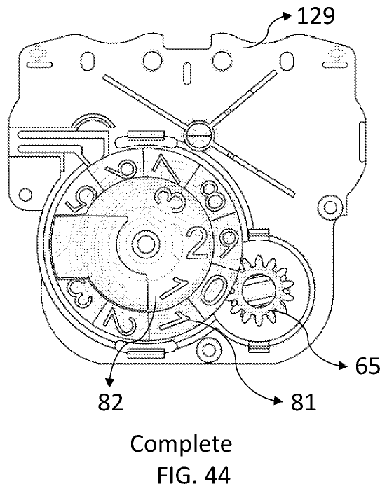

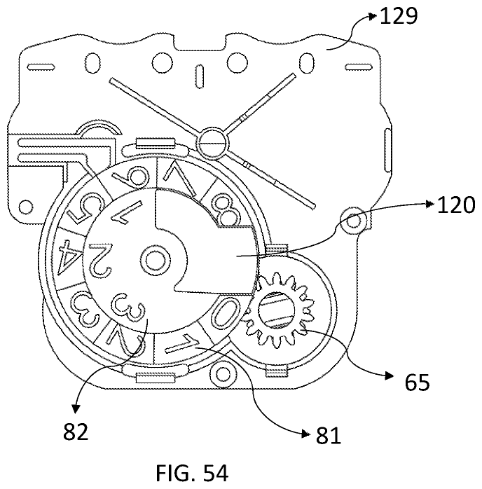

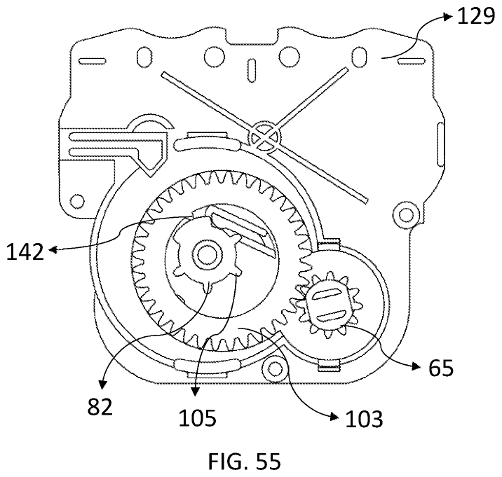

A FIFTH embodiment of the dose counter assembly is shown in FIGS. 40 to 55 of the accompanying drawings.

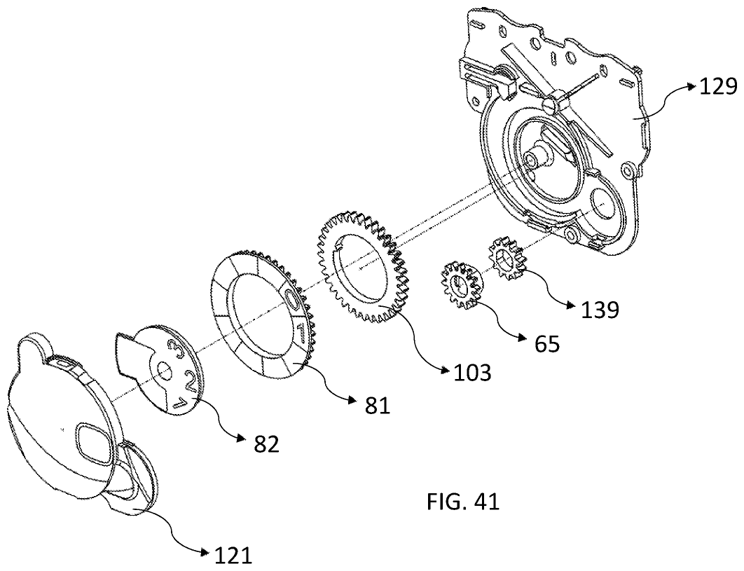

FIG. 40 shows a perspective view of the dose counter assembly. The units wheel (81), the tens wheel (82) and the driver gear (65) are located on the mount plate (129). FIG. 41 shows an exploded view of the dose counter assembly with its components along with the axis of rotation. The components include the units wheel (81), the tens wheel (82), the driver gear (65), the secondary driver gear (139), the transfer wheel (103) and the cover (121). The units dose indicia are disposed on the units wheel (81) and the tens dose indicia are disposed on the tens wheel (82). The units wheel (81) and the tens wheel (82) can rotate at the first axis of rotation, the transfer wheel (103) can rotate at the second axis of rotation, and the driver gear (65) and secondary driver gear (139) can rotate at the third axis of rotation.

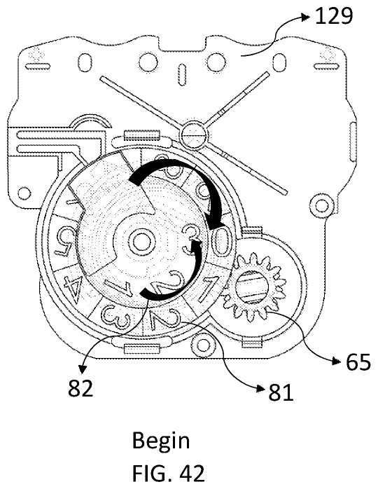

FIGS. 42 to 44 show working of the dose counter assembly and the movement of the dose indicia according to another embodiment of the present disclosure in sequence of begin, in-motion and complete. The units wheel (81) and the tens wheel (82) are concentric and rotate about the post (134). The driver gear (65) rotates and increments the units wheel (81) during each actuation of the dispenser. The tens wheel (82) can be driven by the transfer wheel (103). The transfer wheel (103) can be driven by the secondary driver gear (139) located co-axially with the driver gear (65). The secondary driver gear teeth (140) are constantly in meshed relationship with the transfer wheel (103). The transfer wheel driver tooth (142) drives the tens wheel (82) during every ten actuations of the dispenser. The shutter (149) and the colored portion (148) are located on the tens wheel (82). The shutter (149) remains in the viewing window (48) when the dose count reaches ‘00’.

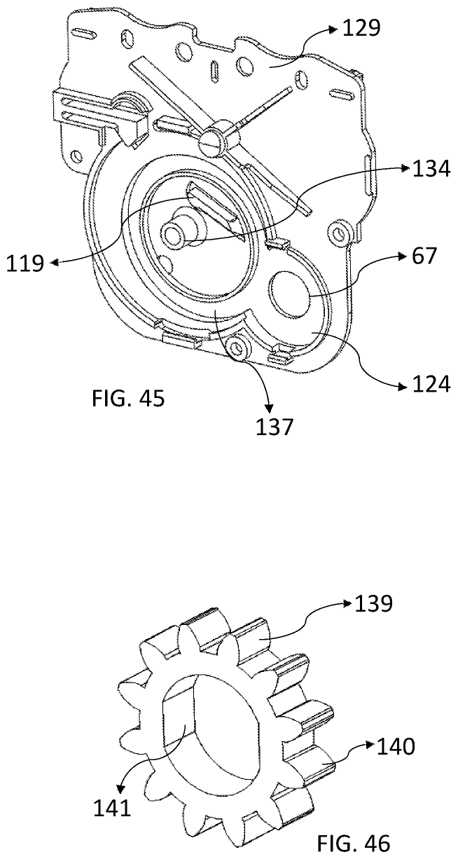

FIG. 45 illustrates an aspect of the mount plate (129). The components of the dose counter assembly can be located on the mount plate (129). The pawl (119) can be integral to the mount plate (129) and prevents the reverse rotation of the tens wheel (82). The opening (67) allow the first waste collector pins (60) to pass through the mount plate (129) and rotate the driver gear (65) via the engaging slots (112). The recess (124) can locate the driver gear (65). The transfer wheel (103) can rotate within the recess (137). The units wheel (81) and the tens wheel (82) can be concentric and rotate about the post (134).

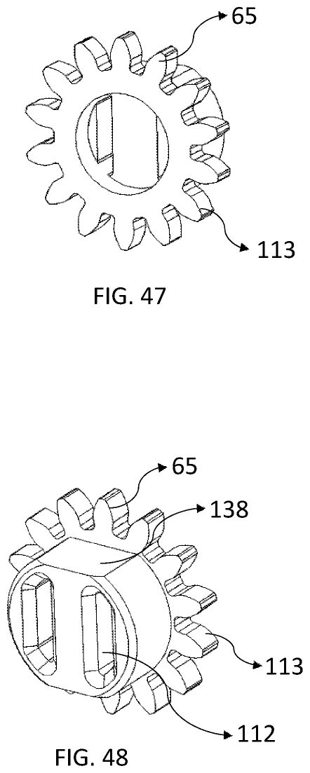

FIG. 46 illustrates an aspect of the secondary driver gear (139). The secondary driver gear (139) can be located co-axially to the driver gear (65). The driver gear (65) drives the secondary driver gear (139) via the flat ends (141). The secondary driver gear teeth (140) are constantly in meshed relationship with the transfer wheel (103).

FIGS. 47 and 48 illustrate the topside and underside of the driver gear (65) according to another aspect of the disclosure. The driver gear (65) which may include teeth (113) and engaging slots (112). The driver gear (65) can be driven by the first waste collector pins (60) via the driver gear engaging slots (112). The driver gear (65) rotates in one direction during each complete actuation of the dispenser and does not rotate in the opposite direction when the dispenser is being closed after use. The unit wheel teeth (104) can always be in a meshed relationship with the driver gear teeth (113). The units wheel (81) can be driven by the driver gear via teeth (113). The flat end (138) of the driver gear (65) mates with and the flat ends (141) of the secondary driver gear (139) and thereby both drive synchronously.

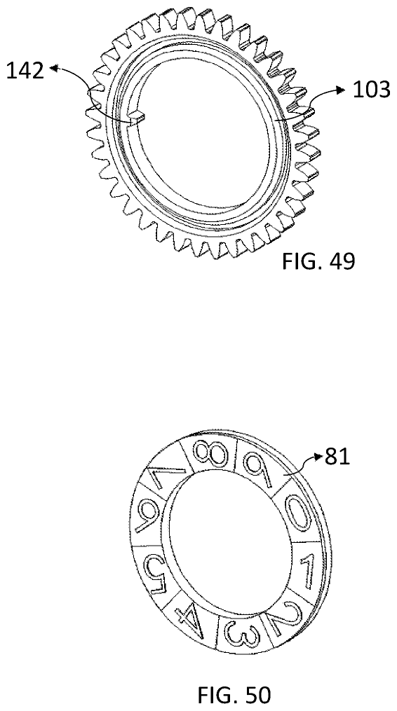

FIG. 49 illustrates as aspect of the transfer wheel (103). The transfer wheel (103) can be located eccentric to the units wheel (81) and the tens wheel (82). The driver tooth (142) of the transfer wheel (103) comes in contact with the tens wheel (82) during every ten actuations of the dispenser and thereby decrements the tens wheel count by one.

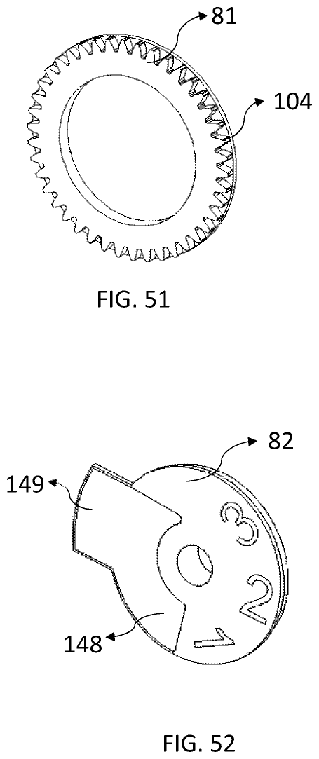

FIGS. 50 and 51 illustrate the topside and underside of the units wheel (81) according to another aspect of the disclosure. The units dose indicia are disposed on the front side of the units wheel (81). The units wheel (81) may include teeth (104) circumferentially on its underside surface. The units wheel teeth (104) are constantly in meshed relationship with the driver gear teeth (113). The driver gear (65) rotates during each actuation of the dispenser and thereby rotates the units wheel (81). The units wheel (81) rotates every discrete angle during each actuation of the dispenser to display the units integer of the dose in the viewing window.

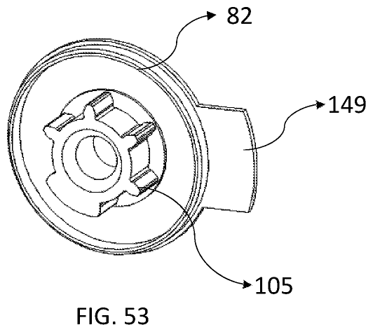

FIGS. 52 and 53 illustrate the topside and underside of the tens wheel (82) according to another aspect of the disclosure. The tens dose indicia are disposed on the topside of the tens wheel (81). The tens wheel (82) may also include the shutter (149) and the colored portion (148) on its topside, and teeth (105) on the underside. The tens wheel (82) can be located concentrically with the units wheel (81). The transfer wheel tooth (142) drives the tens wheel (82) via teeth (105). Due to the eccentric location of the transfer wheel (103), the driver tooth (142) engages with the teeth (105) of the tens wheel (82) during every ten actuations of the dispenser. The shutter (149) is integral to the tens wheel (82) and appear in the viewing window when the dose count reaches ‘00’.

FIGS. 52 to 55 show an aspect of the end position of the dose counter assembly. After the end of dispenser life when all doses are exhausted, the shutter (149) appears in the viewing window (48). At this position, the units wheel (81) and the transfer wheel (103) continue to rotate. The transfer wheel driver tooth (142) is disengaged with the tens wheel tooth (105) due to the eccentric location of the transfer wheel (103). As a result, the tens wheel (82) does not rotate and the shutter (149) remains in the viewing window (48) even if the dispenser is actuated.

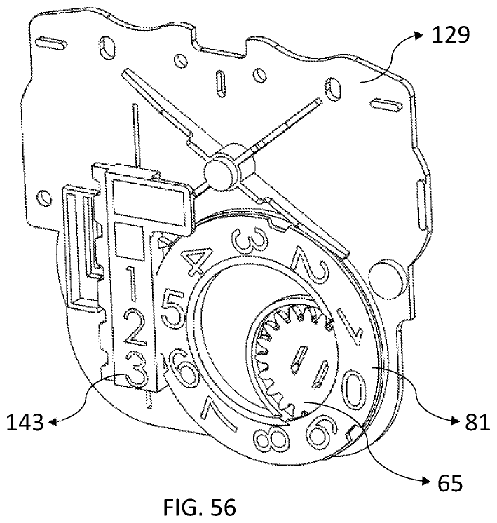

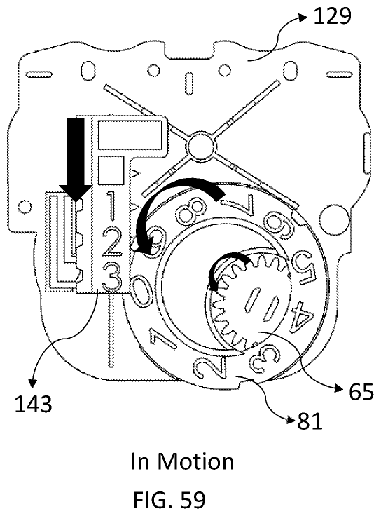

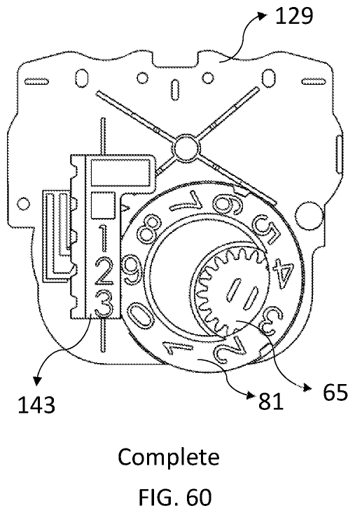

A SIXTH embodiment of the dose counter assembly is shown in FIGS. 56 to 66 of the accompanying drawings.

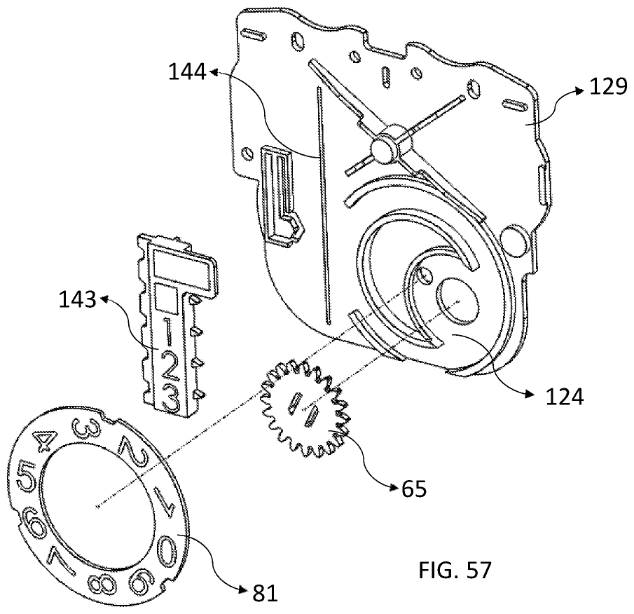

FIG. 56 shows a perspective view of the dose counter assembly. The units wheel (81), the tens slider (143) and the driver gear (65) are located on the mount plate (129). FIG. 57 shows an exploded view of the dose counter assembly with its components along with the axis of rotation. The components include the units wheel (81), the tens slider (143) and the driver gear (65). The mount plate (129) can include a recess (124) and a rail (144). The units dose indicia are disposed on the units wheel (81) and the tens dose indicia are disposed on the tens slider (143). The units wheel (81) can move rotationally at the first axis of rotation and the driver gear can move rotationally at the second axis of rotation. The tens slider (143) can move linearly.

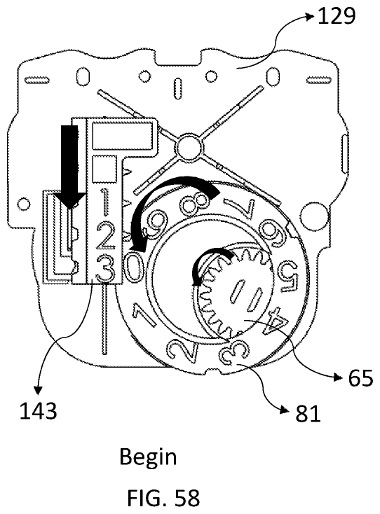

FIGS. 58 to 60 show working of the dose counter assembly and the movement of the dose indicia according to sixth embodiment of the present disclosure in sequence of begin, in motion and complete. The driver gear (65) rotates and increments the units wheel (81) during each actuation of the dispenser. The tens slider (143) can be driven by the units wheel (81) during every ten actuations of the dispenser. The tens slider (143) can be supported and guided by the rail (144) on the mount plate (129). The units wheel (81) and tens slider (143) collectively display the dose count on the viewing window (48) of the dispenser. The shutter (149) and the colored portion (148) are located on the tens slider (143). The shutter (149) remains in the viewing window (18) when the dose count reaches ‘00’. The pawl (119) prevents the reverse rotation of the tens slider (143).

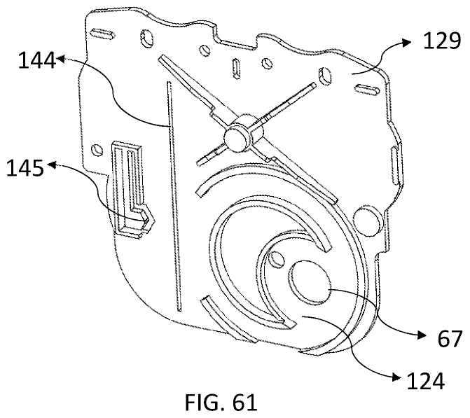

FIG. 61 illustrates an aspect of the mount plate (129). The components of the dose counter assembly can be located on the mount plate (129). The pawl (119) can be integral to the mount plate (129) and prevents the reverse rotation of the tens slider (143). The opening (67) allow the first waster collector pins (66) to pass through the mount plate (129) and rotate the driver gear (65) via the engaging slots (112). The recess (124) can locate the driver gear (65). The tens slider (143) can be supported and guided by the rail (144) of the mount plate (129).

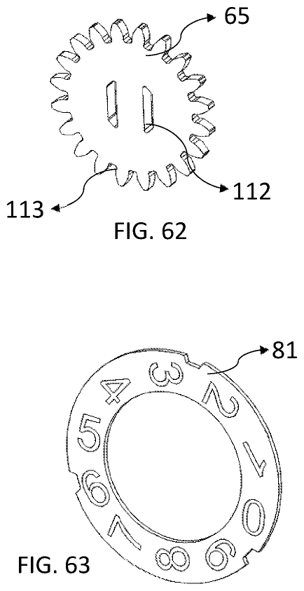

FIG. 62 illustrates the driver gear (65) which may include teeth (113) and engaging slots (112). The driver gear (65) can be driven by the first waste collector (11) (see FIG. 80) via the engaging slots (112). The driver gear (65) rotates in one direction during each complete actuation of the dispenser and does not rotate in the opposite direction when the dispenser is being closed after use. The unit wheel teeth (104) can be continuously in a meshed relationship with the driver gear teeth (113). The units wheel (81) can be driven by the driver gear via teeth (113).

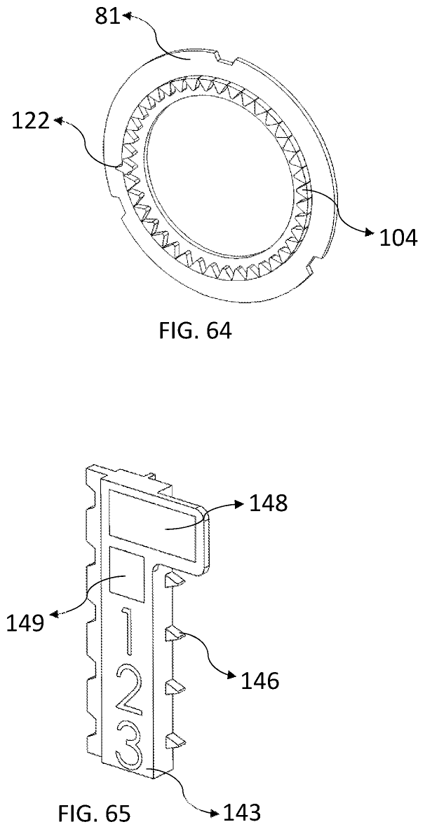

FIGS. 63 and 64 illustrate the topside and underside of the units wheel (81) according to another aspect of the disclosure. The units dose indicia are disposed on the topside of the units wheel (81). The units wheel (81) may include teeth (104) circumferentially and the driver tooth (122) on its underside surface. The teeth (104) and the driver tooth (122) may be extended in the opposite direction. The driver gear (65) rotates during each actuation of the dispenser and thereby rotates the units wheel (81). The units wheel (81) rotates every discrete angle during each actuation of the dispenser to display the units integer of the dose in the viewing window. The units wheel driver tooth (122) meshes with the tens slider teeth (146) during every ten complete actuations of the dispenser.

FIG. 65 illustrates an aspect of the tens slider (143) according to another aspect of the disclosure. The tens dose indicia are disposed on the front side of the tens slider (81). The tens slider (143) may also include the shutter (149) and the colored portion (148) on its front side and teeth (146) extending on the right side surface. The tens slider (143) moves linearly over the rail (144) of the mount plate (129). The tens slider (143) can be driven by the driver tooth (122) of the units wheel (81) during every ten actuations of the dispenser. The shutter (149) can be integral to the tens slider (143) and appears in the viewing window (48) when the dose count reaches ‘00’.

When the user actuates the dispenser, the driver gear (65) drives and increments the units wheel (81). After every ten actuations of the dispenser, the units wheel driver tooth (122) meshes with tens slider teeth (146) and rotate the tens slider (143).

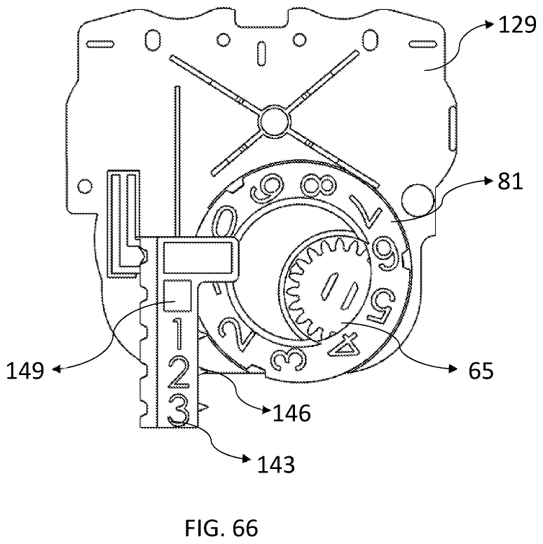

FIG. 66 shows an aspect of the locking mechanism of the dose counter and the dispenser. After the end of dispenser life when all doses are exhausted, the driver tooth (122) of the units wheel (81) no longer meshes with the tens slider teeth (146). As a result, the tens slider (143) does not move and the shutter (149) remains in the viewing window even though the dispenser is actuated.

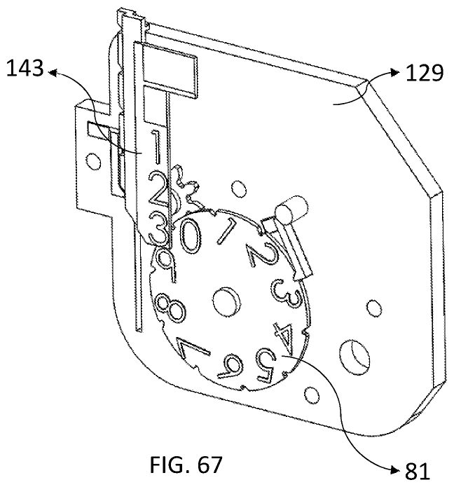

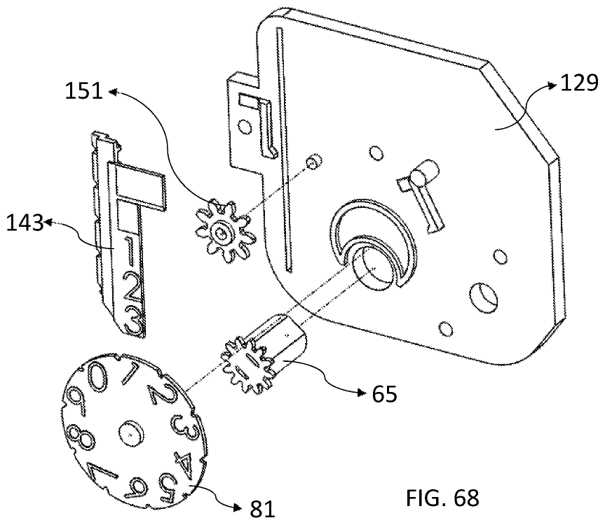

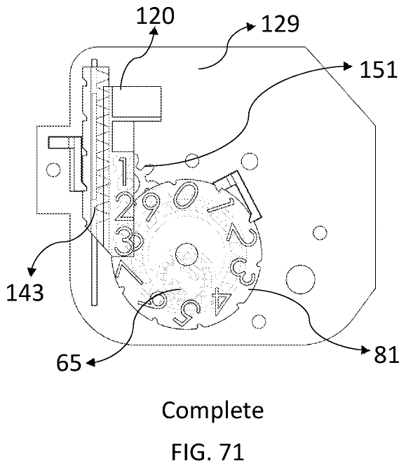

A SEVENTH embodiment of the dose counter assembly is shown in FIGS. 67 to 77 of the accompanying drawings.

FIG. 66 shows a perspective view of the dose counter assembly. The units wheel (81) and the tens slider (143) are located on the mount plate (129). FIG. 67 shows an exploded view of the dose counter assembly with its components along with the axis of rotation. The components include the units wheel (81), the tens slider (143), the driver gear (65) and the idler gear (151). The units dose indicia are disposed on the units wheel (81) and the tens dose indicia are disposed on the tens slider (143). The units wheel (81) can move rotationally at the first axis of rotation, the idler gear (151) can move rotationally at the second axis of rotation, and the driver gear (65) can move rotationally at the third axis of rotation. The tens slider (143) can move linearly.

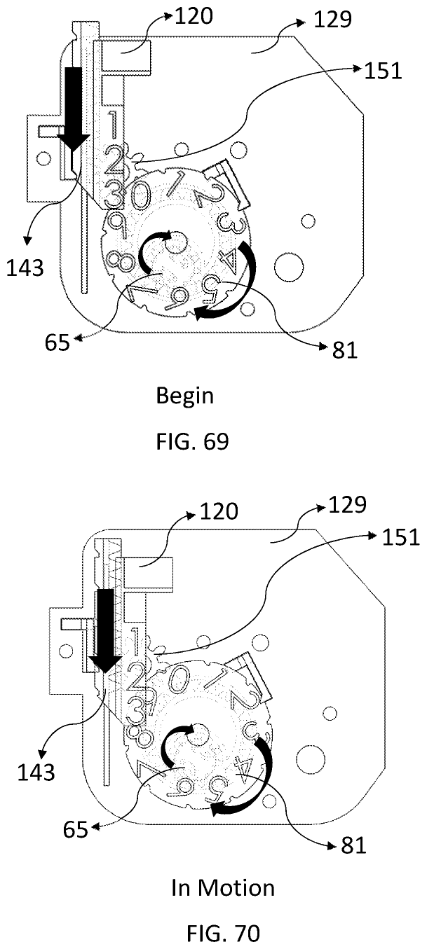

FIGS. 69 to 71 show working of the dose counter assembly and the movement of the dose indicia according to seventh embodiment of the present disclosure in sequence of begin, in-motion and complete. The driver gear (65) rotates and increments the units wheel (81) during each actuation of the dispenser. The tens slider (143) can be driven by the units wheel (81) via idler gear (151) during every ten actuations of the dispenser. The tens slider (143) can be supported and guided by the rail (144) on the mount plate (129). The units wheel (81) and tens slider (143) collectively display the dose count in the viewing window (48) of the dispenser. The shutter (149) and the colored portion (148) can be located on the tens slider (143). The shutter (149) remains in the viewing window (48) when the dose count reaches ‘00’. The pawl (119) prevents the reverse rotation of the tens slider (143).

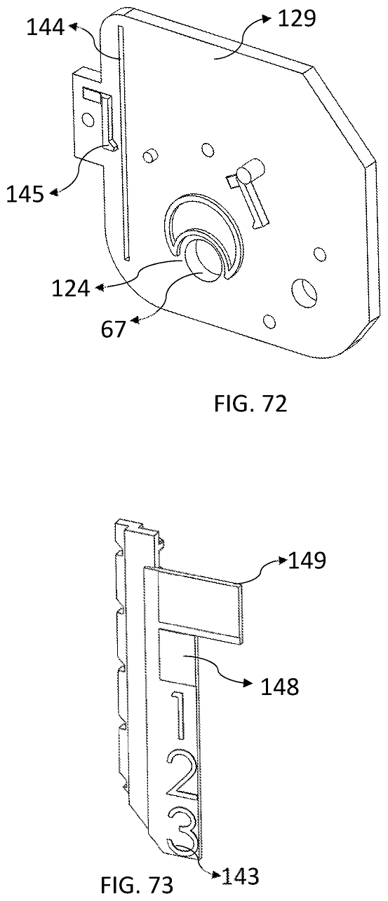

FIG. 72 illustrates an aspect of the mount plate (129). The components of the dose counter assembly can be located on the mount plate (129). The pawl (119) can be integral to the mount plate (129) and prevents the reverse rotation of the tens slider (143). The opening (67) allow the first waster collector pins (66) to pass through the mount plate (129) and rotate the driver gear (65) via the engaging slots (112). The recess (124) can locate the driver gear (65). The tens slider (143) can be supported and guided by the rail (144) of the mount plate (129).

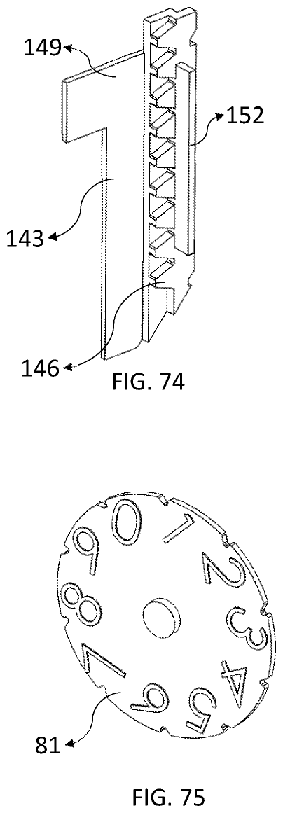

FIGS. 73 and 74 illustrate the topside and underside of the tens slider (143) according to another aspect of the disclosure. The tens dose indicia are disposed on the topside of the tens slider (81). The tens slider (143) may also include the shutter (149) and the colored portion (148) on its topside, and teeth (146) and the guide (152) on the underside. The guide (152) guides the tens slider (143) in linear motion within the rail (144) of the mounting plate (129). The tens slider (143) moves linearly over the rail (144) of the mount plate (129). The tens slider (143) can be driven by the driver tooth (122) of the units wheel (81) via idler gear (151) during every ten actuations of the dispenser. The shutter (149) can be integral to the tens slider (143) and appears in the viewing window when the dose count reaches ‘00’.

The driver gear (65) which may include teeth (113) and engaging slots (112). The driver gear (65) can be driven by the first waste collector (11) (see FIG. 80) via the driver gear engaging slots (112). The driver gear (65) rotates in one direction during each complete actuation of the dispenser and does not rotate in the opposite direction when the dispenser is being closed after use. The unit wheel teeth (104) can be continuously in a meshed relationship with the driver gear teeth (113). The units wheel (81) can be driven by the driver gear via gear teeth (113).

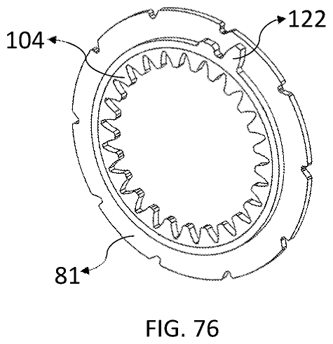

FIGS. 75 and 76 illustrate the topside and underside of the units wheel (81) according to another aspect of the disclosure. The units dose indicia are disposed on the topside of the units wheel (81). The units wheel (81) may include teeth (104) circumferentially and the driver tooth (122) on its underside surface. The teeth (104) and the driver tooth (122) may be extended in the opposite direction. The driver gear (65) rotates during each actuation of the dispenser and thereby rotates the units wheel (81). The units wheel (81) rotates every discrete angle during each actuation of the dispenser to display the units integer of the dose in the viewing window. The units wheel driver tooth (122) meshes with the idler gear (151) which in turn rotates the tens slider teeth (146) during every ten complete actuations of the dispenser.

When the user actuates the dispenser, the driver gear (65) drives and increments the units wheel (81). After every ten actuations of the dispenser, the units wheel driver tooth (122) meshes with tens slider teeth (146) via the idler gear (151) and rotates the tens slider (143).

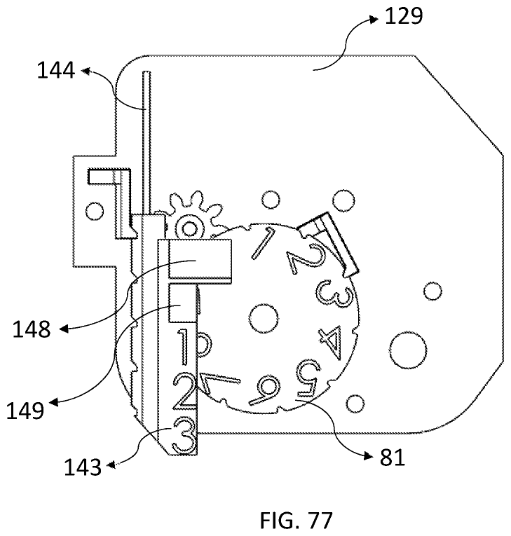

FIG. 77 shows an aspect of the locking mechanism of the dose counter and the dispenser. After the end of dispenser life when all doses are exhausted, the guide (152) of the tens slider (143) bottoms out at the end of the rail (144) of the mount plate (129). As a result, the tens slider (143) does not move and the shutter (149) remains in the viewing window (48) even though the dispenser is actuated.

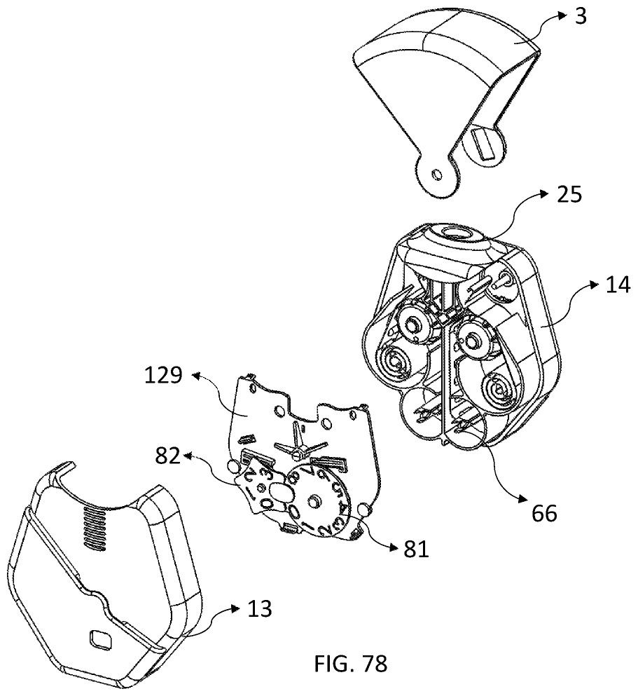

FIG. 78 shows a partially exploded view of the medicament dispenser according to one embodiment of the disclosure. As shown, the medicament dispenser can comprise a top cover (13), a mount plate (129) which can incorporate the dose counter assembly, a base cover (14) which in this embodiment is shown as comprising components of the dispensing mechanism and a mouthpiece opening (25), and a mouthpiece cover (3). FIG. 78 further shows one or more pins (66) of the first waster collector (11).

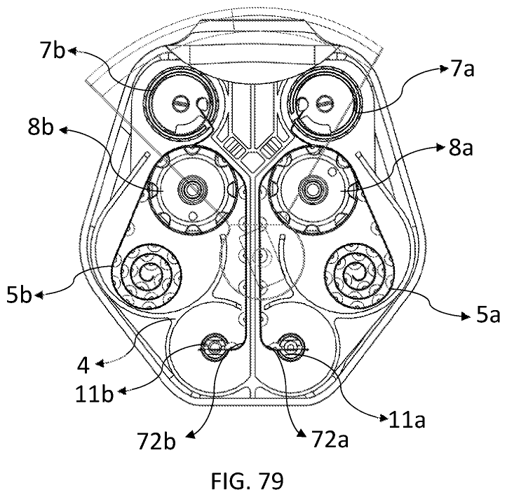

FIG. 79 shows a topside view of the dispensing mechanism and first and second medicament carriers (5a) and (5b). The first and second medicament carriers (5a) and (5b) are positioned within respective left and right compartments of a inner housing assembly (4). Each medicament carrier (5a) and (5b) engages with respective carrier engagements (8a) and (8b). One carrier may contain one or more different medicament(s) than the other carrier. At an opening location (an opening location is a position within the medicament dispenser wherein the medicament carrier is opened or where an individual dose of medicament is otherwise held within the medicament carrier and positioned for access), the base sheet and the lid sheet parts of each medicament carrier (5a) and (5b) are peelably separable. The resulting empty base sheet (72a) and (72b) coils up in respective compartments. The first wastes collectors (11a) and (11b), for wrapping the base sheets of the two medicament carriers, each anchor the end of each respective base sheet (72a) and (72b) in the compartments. Progressive rotation of each respective waste collectors results in the base sheets (72a) and (72b) being wound up therearound into a tight coil. The rotation of each second waste collectors (7a) and (7b) is coupled to that of the respective carrier engagements (8a) and (8b) such the rotation of the carrier engagements (8a) and (8b) rotates new blister packs of the medicament carriers (5a) and (5b) into the appropriate position within the opening location, the second waste collectors (7a) and (7b) correspondingly winding the lid sheets peeled away from each respective medicament carrier base sheet. The resulting separated lid sheets are wound onto/into the respective waster collectors (7a) and (7b) respectively.

FIG. 80 show the first waste collector (11) according to one aspect of the disclosure. The first waste collector (11) can be driven by the components of the dispensing mechanism of the dispenser. The first waste collector teeth (68) can be driven directly or indirectly by the carrier engagement. The one or more pins (66) of the first waste collector (11) pass through the opening (67) of the mount plate (129) and rotate the driver gear (65) via the engaging slots (112).

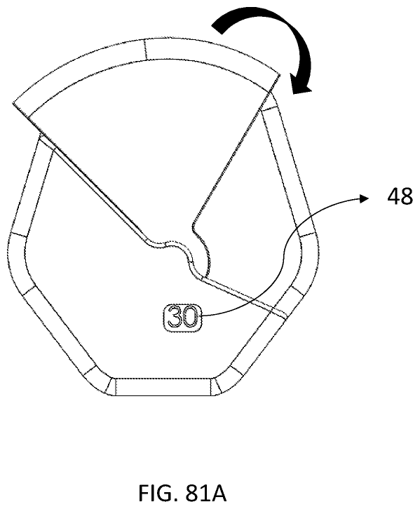

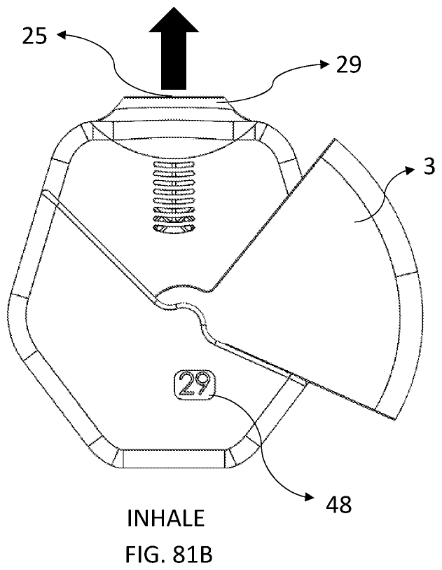

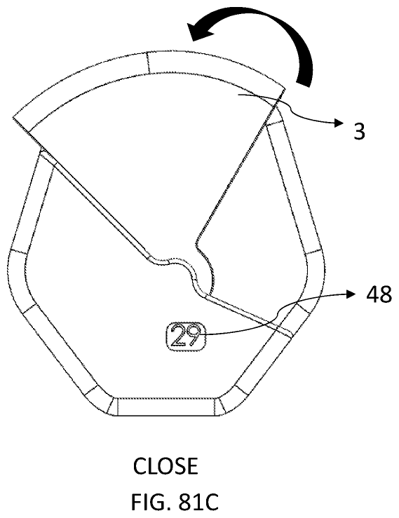

FIGS. 81A and 81C illustrate the demonstration of the advancement of the dose count in the viewing window (48) of the medicament dispenser. In FIG. 80A, the mouthpiece cover (3) is in a closed position in which mouthpiece (29) is not visible. The viewing window (48) displays the dose count indicia reading of ‘30’. In FIG. 80B, the mouthpiece cover (3) has been moved to an opened position in which mouthpiece (29) is visible. As a result of the movement from the closed position to the opened position, the dispensing mechanism (not visible) is actuated in the dispenser to make a medicament dose available for inhalation. The movement has also resulted in the actuation of the dose counter assembly of the dispenser such as to decrease the dose count indicia shown in the viewing window (48) by one unit, here to a new reading of ‘29’. The patient can inhale the medicament through the mouthpiece opening (25). In FIG. 80C, after inhalation, the mouthpiece cover (3) has been returned to the closed position (as in FIG. 81A), and the viewing window (48) displays the same reading ‘29’.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Numbers preceded by a term such as “about” or “approximately” include the recited numbers and values that are ±10%.

Although certain embodiments and examples have been described herein, it will be understood by those skilled in the art that many aspects of the medicament dispensers shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

### EXAMPLE EMBODIMENTS

The following example embodiments identify some possible permutations of combinations of features disclosed herein, although other permutations of combinations of features are also possible.

1. A dose counter assembly for use with a medicament dispenser, the dose counter assembly comprising: a) a units dose display means comprising a first count indicia and one or more driver teeth; b) a tens dose display means comprising a second count indicia; and c) a driver, wherein the first count indicia and second count indicia align at a common viewing area to collectively display a count sequence; wherein the units dose display means and the tens dose display means are non-coaxial.

2. The dose counter assembly according to claim 1, wherein at least the units dose display means or the tens dose display means is in the form of a wheel.

3. The dose counter assembly according to claim 2, wherein at least the units dose display wheel or the tens dose display wheel is in the form of a disc or a ring.

4. The dose counter assembly according to claim 3, wherein the units dose display wheel is in the form of a disc.

5. The dose counter assembly according to claim 3, wherein the tens dose display wheel is in the form of a disc.

6. The dose counter assembly according to claim 1, wherein at least the units dose display means or the tens dose display means is non-circular.

7. The dose counter assembly according to claim 6, wherein the tens dose display means is polygonal.

8. The dose counter assembly according to claim 7, wherein the tens dose display means is polygonal with central axis of rotation.

9. The dose counter assembly according to claim 1, wherein at least the units dose display means or tens dose display means is arranged to move rotationally.

10. The dose counter assembly according to claim 1, wherein the units dose display means is arranged to rotate about a first axis of rotation.

11. The dose counter assembly according to claim 1, wherein the tens dose display wheel is arranged to rotate about a second axis of rotation

12. The dose counter assembly according to claim 1, wherein dose counter assembly further comprises a mount plate.

13. The dose counter assembly according to claim 12, wherein the mount plate comprises a locking arm.

14. The dose counter assembly according to claim 1, wherein the tens dose display means further comprises a locking key.

15. The dose counter assembly according to claim 14, wherein the locking key of the tens dose display means engages with the locking arm of the mount plate to lock the dose counter assembly at the end of the count sequence.

16. The dose counter assembly according to claim 1, wherein the tens dose display means is in the form of a slider.

17. The dose counter assembly according to claim 16, wherein the second count indicia are disposed on the tens dose display slider linearly.

18. The dose counter assembly according to claim 16, wherein the tens dose display slider is arranged to move linearly.

19. The dose counter assembly according to claim 18, wherein the tens dose display slider is arranged to move linearly and tangent to the units dose display wheel.

20. The dose counter assembly according to claim 16, wherein the tens dose display slider further comprises a set of teeth.

21. The dose counter assembly according to claim 20, wherein the teeth of the tens dose display slider disengage with one or more driver teeth at the end of the count sequence.

22. The dose counter assembly according to claim 1, wherein the driver is arranged to rotate about a third axis of rotation.

23. The dose counter assembly according to claim 22, wherein the driver is in meshed relationship with the units dose display means.

24. The dose counter assembly according to claim 23, wherein the driver is adapted to couple with a dispensing mechanism of the medicament dispenser.

25. The dose counter assembly according to claim 1, wherein the tens dose display means comprises a set of teeth.

26. The dose counter assembly according to claim 25, wherein one or more driver teeth of the unit dose display means is arranged for intermittent meshing with the teeth of the tens dose display means.

27. The dose counter assembly according to claim 26, wherein the tens dose display means further comprises an end location wherein the teeth are absent.

28. The dose counter assembly according to claim 27, wherein the teeth of the tens dose display means disengage with one or more driver teeth at the end location.

29. The dose counter assembly according to claim 1, wherein the tens dose display means further comprises a shutter.

30. The dose counter assembly according to claim 29, wherein the shutter appears at the common viewing area at the end of the count sequence.

31. The dose counter assembly according to claim 30, wherein the units dose display means continues to rotate without rotating the tens dose display means at the end of the count sequence.

32. The dose counter assembly according to claim 12, wherein the mount plate further comprises a pawl.

33. The dose counter assembly according to claim 32, wherein the pawl prevents reverse movement of the tens dose display means.

34. A medicament dispenser incorporating the dose counter assembly according to claim 1.
